(19) United States
(12) Patent Application Publication
(10) Pub. No.: US 2024/0263143 A1
(43) Pub. Date: Aug. 8, 2024

(54) Baby Hamster Kidney (BHK) Cells Transformed with the Adenoviral E1 Gene for Production of Recombinant Adeno-Associated Virus

(71) Applicant: Agathos Biologics, Fargo, ND (US)

(72) Inventors: James Brown, Fargo, ND (US); Michael Chambers, Fargo, ND (US); John Ballantyne, Fargo, ND (US); Jagadish Loganathan, Fargo, ND (US); Andrzej Noyszewski, Fargo, ND (US); Amber Plambeck, Fargo, ND (US)

(73) Assignee: Agathos Biologics, Fargo, ND (US)

(21) Appl. No.: 18/430,090

(22) Filed: Feb. 1, 2024

Related U.S. Application Data
(60) Provisional application No. 63/482,873, filed on Feb. 2, 2023.
(60) Provisional application No. 63/487,759, filed on Mar. 1, 2023.

(51) Int. Cl. C12N 5/071; C12N 7/00; C12N 15/86

(52) U.S. Cl. CPC C12N 5/0686; C12N 5/067; C12N 7/00; C12N 15/86; C12N 2510/02; C12N 2750/14143; C12N 2750/14151

(57) ABSTRACT

Disclosed is the creation of new E1-complementing BHK-21 cell lines to produce recombinant adeno-associated virus (rAAV) vectors. The new cell lines stably express the E1 gene region of adenovirus or a portion thereof and produce the E1 proteins. Transient production in the E1-complementing cell lines of the AAV rep/cap proteins, helper proteins and an AAV transfer plasmid containing the desirable transgene flanked by the Inverted Terminal Repeat (ITR) sequences of AAV, results in production of rAAV particles containing the transgene. Further disclosed is scaled-up production, harvesting and purification of transgene-containing rAAV. The purified rAAV has demonstrated capability to infect a host cell line and express the protein encoded by the transgene. The disclosure provides non-human, non-embryonic cell lines for production of rAAV particles that are a platform for delivery of a desired transgene.

### RELATED APPLICATIONS

This application claims priority to U.S. Provisional Patent Application No. 63/482,873 filed on Feb. 2, 2023, titled “Baby Hamster Kidney (BHK) Cells Transformed with the Adenoviral E1 Gene for Production of Recombinant Adeno-Associated Virus,” and to U.S. Provisional Patent Application No. 63/487,759 filed on Mar. 1, 2023, titled “Baby Hamster Kidney (BHK) Cells Transformed with the Adenoviral E1 Gene for Production of Recombinant Adeno-Associated Virus,” and the entire contents of each are incorporated herein.

### STATEMENT REGARDING GOVERNMENT FUNDING

This work was funded in part by Grant No. 21-283 from the North Dakota Department of Agriculture's Bioscience Innovation Grant Program.

### SEQUENCE LISTING

An electronic sequence listing (828349-00003.xml; size 35.6 KB; date of creation Jan. 29, 2024) submitted herewith is incorporated by reference in its entirety.

### TECHNICAL FIELD

The invention relates to the development of new cell lines to produce recombinant adeno-associated virus (rAAV) particles that encode and are capable of expressing a transgene.

### BACKGROUND OF INVENTION

Genetic medicine holds great potential for correcting disease-causing defects, targeting and destroying cancerous tissues, and providing speed and flexibility for the development of vaccines. However, the manufacture of genetic treatments and vaccines is very expensive and requires specialized production capacity, which is of limited availability. Recombinant DNA genetic material to be used as a gene therapy or a vaccine is incorporated into a virus-based vector system, such as an adeno-associated virus (AAV), which is produced by expression of the viral vector components in immortalized living cells maintained in tissue culture.

Adeno-associated virus (AAV) vectors are one platform for potential gene delivery for the treatment of a variety of human diseases. There is a need to develop clinically-useful rAAV particles, to optimize genome designs and harness the potential revolutionary biotechnologies that could contribute substantially to the growth of the gene therapy field. Preclinical and clinical successes in AAV-mediated gene replacement and gene editing have helped establish rAAV as a promising therapeutic vector, with four AAV-based therapeutics gaining regulatory approval in Europe or the United States and more in clinical development. Continued study of AAV biology and increased understanding of the associated therapeutic challenges and limitations will build the foundation for future clinical success (see Wang, D., Tai, P. W. L. & Gao, G. Adeno-associated virus vector as a platform for gene therapy delivery. Nat. Rev. Drug. Discov. 18, 358-378 (2019)).

In nature AAV requires co-infection with another virus (a helper virus), typically adenovirus, to propagate. Adenovirus provides the requisite helper functions primarily through expression of its early-region genes (E1, E2, E4 and VA RNA). Use of wild-type adenovirus to supply helper functions for production of rAAV presents complexity and is a safety risk for human administration of the final product if the design of the production could result in a replication-competent adenovirus. Enabling rAAV production without a helper virus, a so-called “helper virus-free” method, is thus desirable. Cell lines that contain genomic E1 genes have been established for helper virus-free production of recombinant adenovirus (rAd), including cell lines derived from human embryonic kidney (HEK293), HeLa (GH329), A549 (SL0003) and human embryonic retina (PER.C6) cells. The need for production of rAAV without a helper virus resulted in development of a method using HEK293 by providing the required helper functions in a helper plasmid, which contains all helper genes necessary for production of rAAV except the E1 gene, which is provided by the HEK293 cell. Transfection of HEK293 with the helper plasmid, a plasmid with the replication and capsid genes of AAV delivered in trans, and a plasmid with a transgene delivered in cis flanked by the inverted terminal repeats (ITRs) that flank the replication and capsid genes in the wild-type AAV genome, results in production of a rAAV particle that contains the transgene and that can infect cells and produce the protein encoded by the transgene.

HEK293 is an immortalized cell line generated in 1973 by transfection of cultures of normal human embryonic kidney cells with sheared adenovirus type 5 (Ad5) DNA, resulting in stable integration of the adenoviral E1 gene into its genome. The previous use of HEK293 as the host cell line for production of therapeutic biologics that are in active clinical trials and other rAAV therapeutics already approved by the FDA, makes production of rAAV in HEK293 a “proven” method that is familiar to regulatory agencies and, consequently, attractive to clinical trial sponsors because they understand the related regulatory requirements. Developing a new E1-complementing cell line that satisfies the regulatory requirements for production of rAAV would be expensive and risky, and consequently the field has focused on improving the performance of HEK293 as a host for rAAV production.

Another adenovirus E1-complementing immortal cell line is PER.C6. PER.C6 is a cell line derived from human embryonic retinal cells transformed with the adenovirus type 5 (Ad5) E1A and E1B genes that was developed for adenovirus vector production via plasmid transfection. It contains a partial E1 sequence, instead of the full wild-type E1 sequence present in HEK293, to avoid formation of replication-competent adenovirus. There are no reports of PER.C6 ever being used to produce rAAV particles, but production of adenovirus resulting from transfection and stable integration of a partial E1 sequence suggests hypothetically that PER.C6 could produce rAAV. The cell line is proprietary and is not commercially available. Use of HEK293 or the potential use of PER.C6 as adenovirus E1-complementing cell lines to produce rAAV for genetic medicine suffers from the ethical concerns regarding the origin of those materials from aborted fetuses. Although HEK293 was established in 1973 and has been used for production of commercial products, it is not clear whether it derived from an aborted fetus, which is considered most likely, or a miscarriage. Additionally, success in gene therapy has increased the demand to produce rAAV at high yield and at large scale and, therefore, new cell lines that meet the requirements to produce commercial products are desirable.

Many of the immortalized cell lines currently available for production of nucleic acid-based gene therapy or vaccine products either lack sufficient history and documented progeny, or clearly originate from aborted human fetal tissue, which results in an ethical dilemma for those who do not wish to use products derived from aborted human fetal tissue. The development of non-aborted human fetal-cell lines has been inhibited by the tendency of drug developers to use cell lines for manufacture of products that were previously approved by the FDA or other regulatory agencies. As stated above, the established use of cell lines from aborted human fetal tissue such as HEK293 for production of recombinant AAV particles means that pharmaceutical manufacturers can leverage existing data to support their use, whereas the manufacturer may have to produce more data when using a new cell line, potentially increasing the cost and time of development. The established data and the properties of cells from aborted fetal tissue that make them amenable to biomanufacturing have the practical effect of limiting the cell lines available to manufacturers, resulting in an ethical dilemma for some consumers.

HEK293 was established in 1973 by harvesting kidney cells from a human embryo that was likely aborted. Cells from embryonic tissue are known to be well-suited for protein expression and bioproduction, and several cell lines and primary cell banks, including PER.C6, WI-38, and MRC-5, were established from aborted human fetal tissue more than 40 years ago and are used for biomanufacturing. As recently as 2015 a new cell line, Walvax-2, was developed from aborted fetal lung tissue and is a candidate host cell line for vaccine production. Many people consider elective abortion to be an immoral act and consider themselves to be indirectly complicit if they use products manufactured using material from an aborted fetus. Some consumers choose not to use those products. Cell lines derived from ethical sources that demonstrate equivalent or improved performance will provide pharmaceutical companies with options for biomanufacturing that eliminate ethical concerns and result in expanded access to vaccines and biopharmaceuticals.

There are two other methods for utilizing the AAV vector system for manufacturing recombinant AAV (rAAV) particles. One uses baculovirus and an insect cell line as the host. Helper functions required for AAV assembly are provided by the baculovirus genome. This is more complex than delivering the necessary viral genes via transfection of plasmids because it involves production of one or more baculoviruses. Another method for producing rAAV particles uses a Herpes Simplex Virus (HSV) vector to deliver the required genes to Baby Hamster Kidney (BHK) cells used as the host. Like the insect cell method, this is more complex than producing rAAV particles using HEK293 because it involves production of one or more recombinant HSV vectors, with helper functions provided by HSV.

Ethically-sourced tissues provide an alternative for those who do not want to use products made using human aborted fetal cell lines. They may originate from fetal tissue (e.g., ectopic pregnancy, spontaneous abortion), differentiated induced pluripotent stem cells (iPSCs) and human trophoblast stem cells (hTSCs), other human tissue, or other mammalian cells. Ethically-sourced cells include those pre-existing or new cell sources such as existing cell lines that could be made E1-complementing to support production or rAd or rAAV. Ethically-sourced cell lines that are candidates for complementation with E1 include BHK, A549, CHO, Vero, HeLa, and other cell lines not derived from electively-aborted fetal tissue. In some instances, additional non-human mammalian sources of cell lines are possible, such as sheep or jackrabbit. Ethically-sourced cells may be adherent or suspension cells. However, no non-human, non-embryonic cell line has been made E1-complementary for production of rAAV vectors and the inherent advantages of embryonic tissue for viral vector production discourages the development of a suitable non-embryonic host cell line and suggests that such development is not likely to succeed.

The present method uses the BHK-21 cell line, which is not human and non-embryonic. BHK-21 was established in 1961 from kidney cells of a one-day old hamster and has been used in production of commercial products, including veterinary vaccines for rabies (see Lalosević, D., Lalosević, V., Lazarević-Ivanc, L. & Knezević, I. BHK-21 cell culture rabies vaccine: immunogenicity of a candidate vaccine for humans. Dev. Biologicals 131, 421-9 (2008)) and foot and mouth disease (see Pay, T. W., Boge, A., Menard, F. J. & Radlett, P. J. Production of rabies vaccine by an industrial scale BHK 21 suspension cell culture process. Dev. Biol. Stand. 60, 171-4 (1985)) and human clotting Factors VIIa and VIII, (see Dumont, J., Euwart, D., Mei, B., Estes, S. & Kshirsagar, R. Human cell lines for biopharmaceutical manufacturing: history, status, and future perspectives. Crit. Rev. Biotechnol. 36, 1110-1122 (2016)) and so is generally regarded as well understood for regulatory purposes. As demand for production of rAAV has grown, there is a growing need for more and alternative cell lines for production and for higher production yields of rAAV than the existing methods and cell lines provide. Consequently, there is a need in the art for a BHK-E1 complementing cell line that can be used to produce rAAV. The BHK-E1 cell lines of the present invention may be used in applications that currently use HEK293 for production of rAAV. These uses include viral vector production, general protein expression and production, and assays to determine the expression of proteins from various constructs and delivery methods. When used under GMP conditions, the BHK-E1 cell lines of the present invention may be used to produce viral vectors and other biologics for administration to humans or other mammals.

### SUMMARY OF THE INVENTION

To make BHK-E1 complementing cell lines for production of rAAV, BHK-21 cells are transfected with a plasmid containing the wild-type sequence of the human adenovirus serotype 5 (HAdV-5) gene (E1) or a portion thereof and a gene coding for resistance to hygromycin, which is an antibiotic that also kills higher eukaryotic cells by inhibiting protein synthesis. After transfection the BHK-21 cells are grown in media that includes hygromycin, which kills any cells that did not take up the plasmid. After several passages the E1 protein is detected via Western blot in the E1-transfected cells compared to control BHK-21 cells that are not transfected. Measurement of E1 expression in the hygromycin-resistant BHK-21 cells is consistent through multiple passages of the cells. The E1-complementing BHK-21 cells are transfected with three plasmids that separately encode a transgene flanked by Inverted Terminal Repeat (ITR) sequences of AAV, AAV rep/cap proteins and helper virus proteins to produce rAAV encoding the transgene. Recombinant AAV is collected and the identity is confirmed by an immunoassay to the viral capsid, quantitative digital PCR measurement of the transgene, and Western blot detection of the three proteins comprising the rAAV capsid—VP1, VP2 and VP3. The E1-complementing BHK-21 cells of the present invention produce rAAV particles of any AAV serotype including serotypes 2, 5, 6 and 8. Production of rAAV particles containing a transgene is scaled-up to produce rAAV for infectivity and production of the protein encoded by the transgene. The rAAV containing the transgene is harvested, purified and used to reinfect an appropriate host cell line resulting in expression of the transgene and production of the polypeptide encoded by the transgene.

The E1 gene used to make the E1-complementing BHK-21 cell line may be the wild-type E1 region of any Adenovirus serotype. In some instances, the E1 gene could be a portion of an adenovirus E1 region. The E1 region could vary from wild type in its nucleotide sequence or number of bases if it results in an E1-complementing BHK-21 cell line when integrated into the genomic DNA of the cell line.

In one embodiment, the E1 gene used to make the E1-complementing BHK-21 cell line may be the wild-type E1 region (bp 1 to 4344) of human adenovirus 5 (hAd5) (SEQ ID NO: 1). In another embodiment, the functional E1 gene used to make the E1-complementing BHK-21 cell line is a nucleic acid sequence having at least 90% sequence identity with the wild-type E1 region (bp 1 to 4344) of human adenovirus 5 (hAd5) (SEQ ID NO: 1). In a further embodiment, BHK-21 cells are transfected with a plasmid containing an abbreviated sequence of the human adenovirus serotype 5 (HAdV-5) gene region (bp 560-3509) (SEQ ID NO: 2) (E1AE1BbGH) with a human phosphoglycerate kinase promoter (HuPGK), a Kozak consensus sequence (a motif to enhance recognition of the protein translation initiation site) and a gene coding for resistance to hygromycin. The E1AE1BbGH construct is made by removing the Ad5 ITR region up to the region of ATG of E1A CDS (coding sequence) and replacing it with the sequence for the HuPGK promoter and a Kozak sequence. Sequences downstream from the E1A CDS including those coding for E1B, pIX and part of pIVa2, all of which are not modified from the original Ad5 sequences, are followed by a bovine growth hormone polyadenylation (bGH-poly(A)) signal. In a further embodiment, the E1 region used to make the cell line is a portion of human adenovirus serotype 5 (HAdV-5) gene region (bp 560-3509) (SEQ ID NO: 2), for example a nucleotide sequence having at least 90% sequence identity with an abbreviated sequence of the human adenovirus serotype 5 (HAdV-5) gene region (bp 560-3509) (SEQ ID NO: 2).

In some instances, the expression of the E1 gene region may be modified using any appropriate promoter, consensus or polyA sequences. Any selectable marker appropriate for selection in mammalian cells may be used. The invention is not limited to the use of hygromycin. In some instances, the E1 gene may be incorporated into the BHK-21 cells by any appropriate method including transfection of BHK-21 cells with sheared adenovirus DNA, gene editing or transposon insertion. The invention is not limited to transfection of BHK-21 with a plasmid containing a portion of the E1 gene region and a selectable marker. The E1-complementing BHK-21 cell line may be a recombinant polyclonal cell line or a monoclonal cell line. A monoclonal line can be established by picking clones or by any other method known in the art.

For production of rAAV particles encoding a transgene, the host E1-complementing cell line can be provided with a transgene flanked by Inverted Terminal Repeat (ITR) sequences of AAV, AAV rep/cap proteins and helper virus proteins by any method known to the person of skill in the art. Those genes can be incorporated in the genome of the cell line or transiently present on one, two or three vectors, such as plasmids, or other exogenous DNA. In one embodiment, E1-complementing BHK-21 cells are transfected with three plasmids that separately encode a transgene flanked by Inverted Terminal Repeat (ITR) sequences of AAV, AAV rep/cap proteins and adenovirus helper virus proteins to produce rAAV. The AAV rep/cap proteins are AAV serotype 2, AAV serotype 5, AAV serotype 6, AAV serotype 8, a naturally-occurring serotype, an artificial serotype, or a combination of two or more of the foregoing.

The rAAV particles produced in the present invention may be used to infect any appropriate host cell line. The host cell line may be animal cells including human cells. In one embodiment, harvested and purified rAAV particles containing a transgene are used to infect HepG2 cells and expression of the polypeptide encoded by the transgene is demonstrated. The transgene of the present invention may be any suitable gene that encodes a polypeptide, including a therapeutic gene or therapeutic polypeptide providing benefit to an animal including a human patient. In some embodiments, the therapeutic gene or polypeptide may be used for gene therapy or a vaccine correcting disease-causing defects, targeting and destroying cancerous tissues, gene delivery for treatment of human disease, preclinical and clinical AAV-mediated gene replacement and gene editing as a therapeutic vector. In some embodiments, the transgene is luciferase. In other embodiments, the transgene is green fluorescent protein (GFP). In other embodiments, rAAV2-luciferase and rAAV8-luciferase particles are harvested, purified and used to infect HepG2 cells and the production of the transgene luciferase is demonstrated.

### DETAILED DESCRIPTION OF THE DRAWINGS

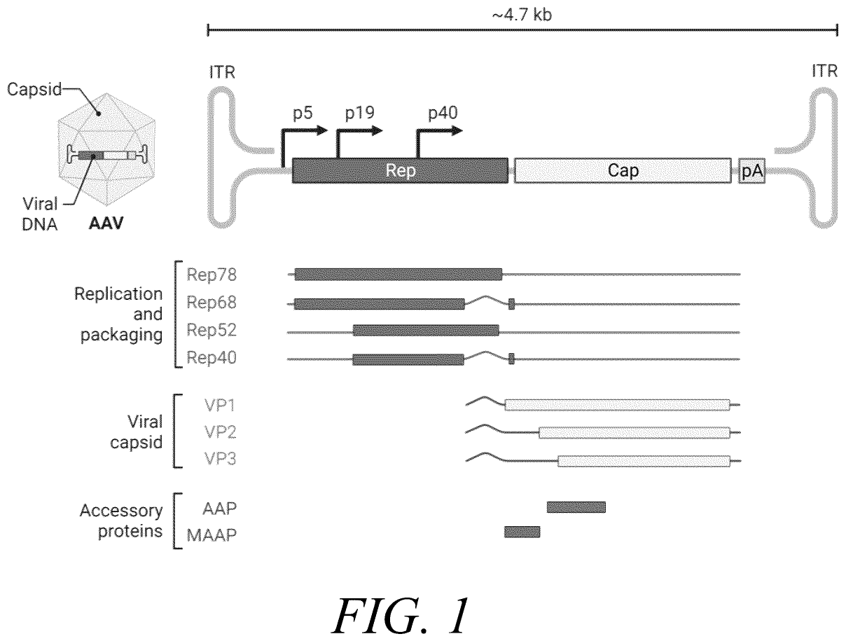

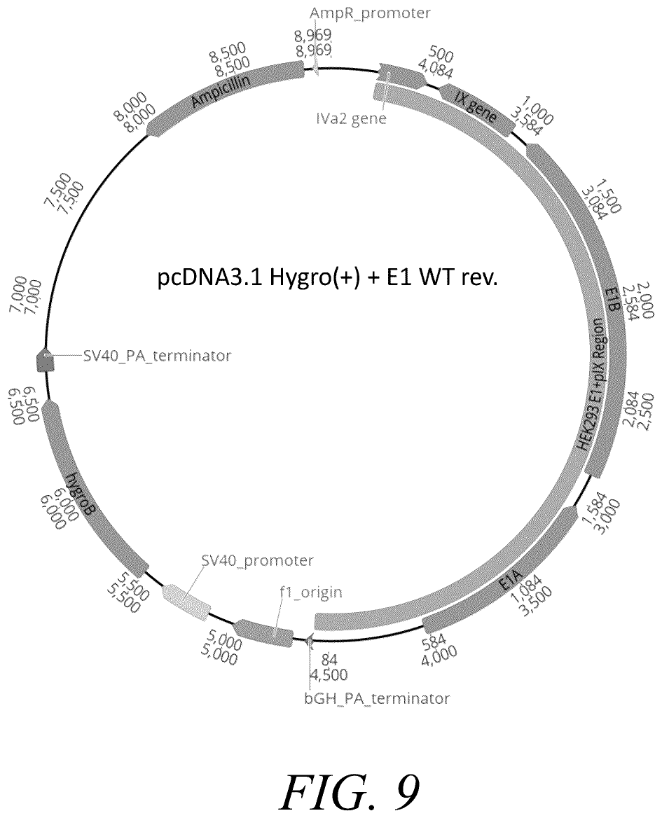

The present disclosure can be better understood, by way of example only, with reference to the following drawings. The elements of the drawings are not necessarily to scale relative to each other, emphasis instead being placed upon clearly illustrating the principles of the disclosure. FIGS. 1-8 were created with Biorender.com. FIGS. 9-11 were created with Geneious version 2023.0.4. Statistical analysis and bar graphs were made with GraphPad Prism Version 9.5.1.

FIG. 1 is a schematic representation of the wild-type adeno-associated virus (AAV) genome having replication and packaging, capsid, and accessory protein genes.

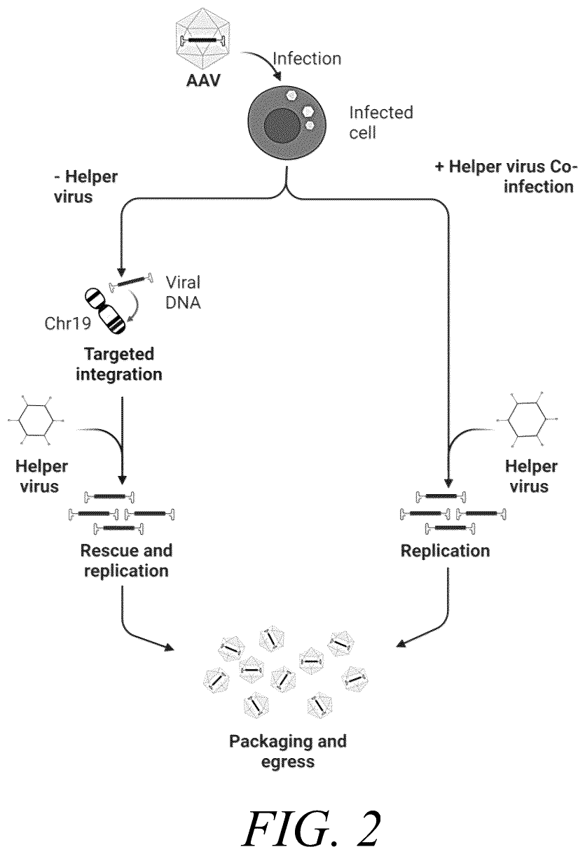

FIG. 2 is a schematic representation of the AAV lifecycle, which requires co-infection with a helper virus.

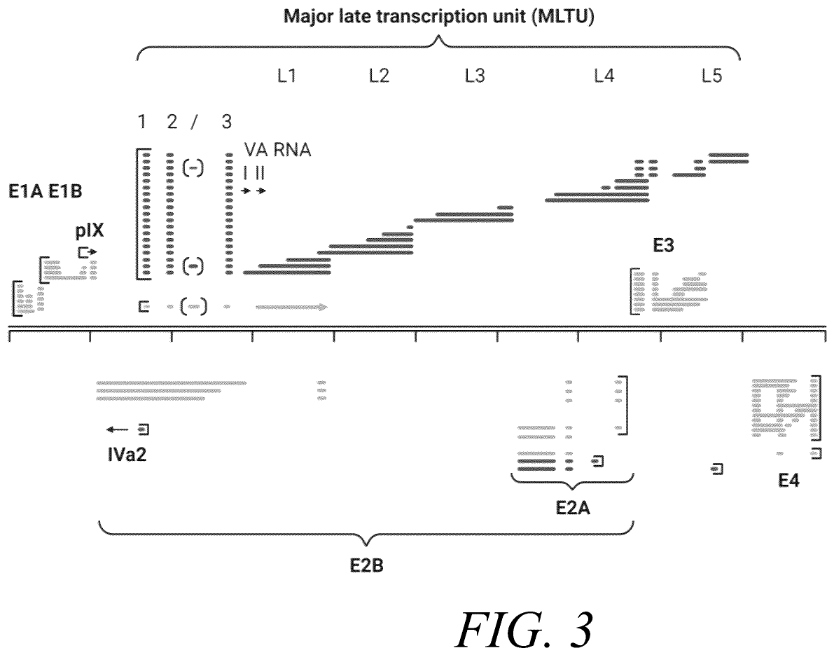

FIG. 3 is a schematic representation of a linearized adenovirus genome including helper genes for AAV propagation.

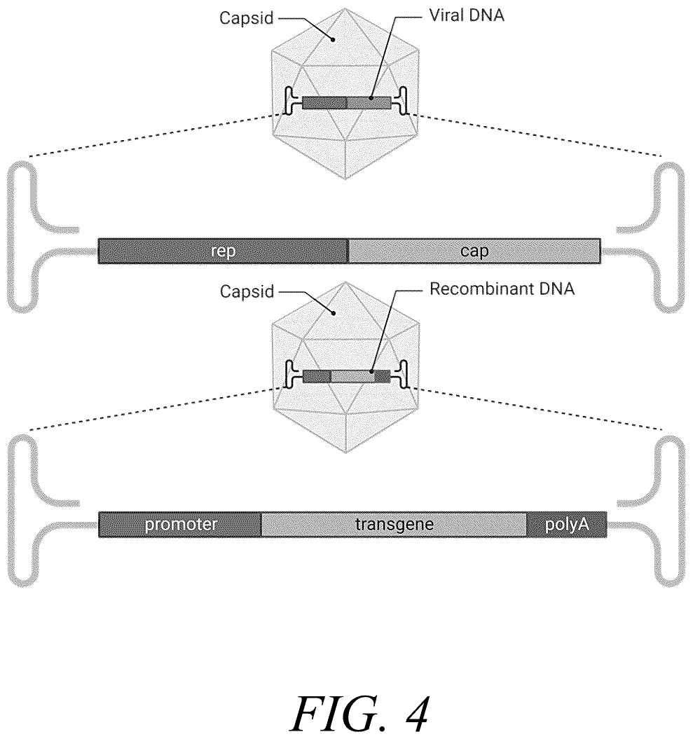

FIG. 4 is a schematic representation of linearized wild-type AAV with rep and cap genes (top panel) and recombinant AAV with a promoter, transgene and poly A region replacing the rep and cap genes (lower panel).

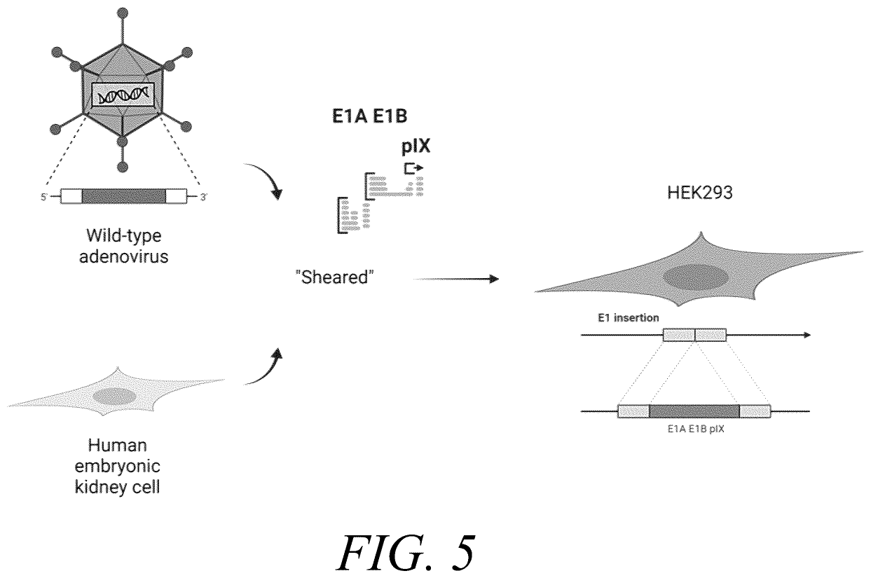

FIG. 5 is a schematic representation of the engineering of HEK293 cells to integrate adenovirus E1 genes into its genome.

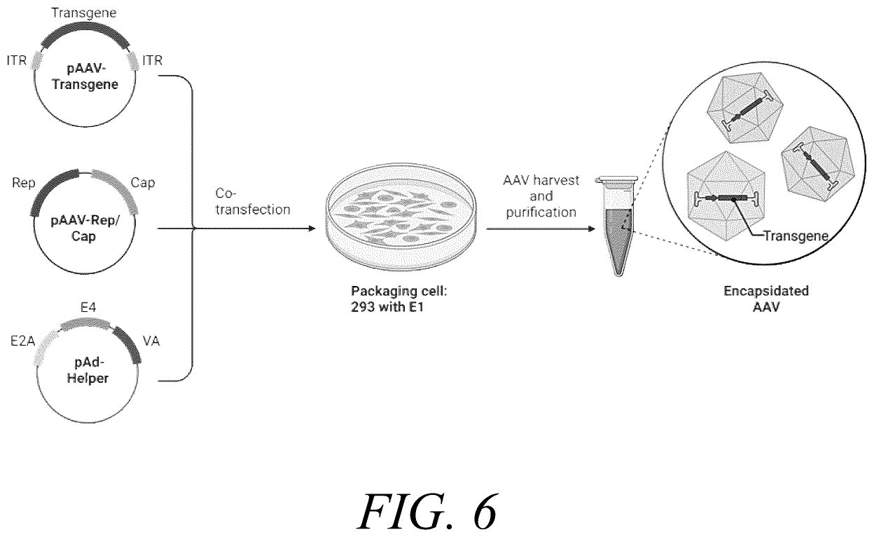

FIG. 6 is a schematic representation of recombinant AAV production via triple transfection of HEK293 cells with plasmids separately containing the transgene, adenovirus helper genes, and AAV rep/cap genes.

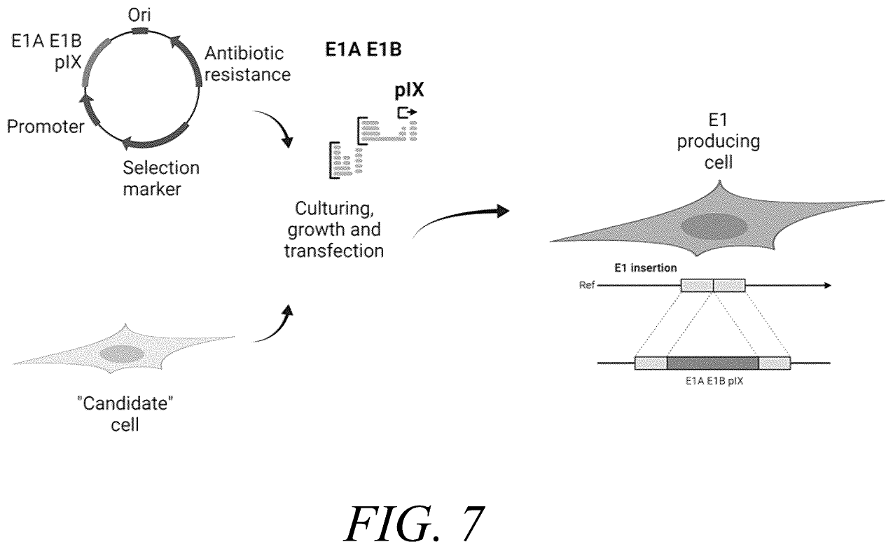

FIG. 7 is a schematic representation of the transformation of a mammalian cell by transfection with a plasmid containing the adenovirus E1 genes and a gene for resistance to a selectable marker to create an E1-complementary mammalian cell. The selectable marker may include a gene that confers resistance to hygromycin, neomycin, puromycin, or another appropriate antibiotic. The E1 gene will insert within a chromosome of the cell. In HEK293, the E1 gene is located at human chromosome 19 (19q13.2). See Louis, N., Evelegh, C. & Graham, F. L. Cloning and Sequencing of the Cellular-Viral Junctions from the Human Adenovirus Type 5 Transformed 293 Cell Line. Virology 233, 423-429 (1997).

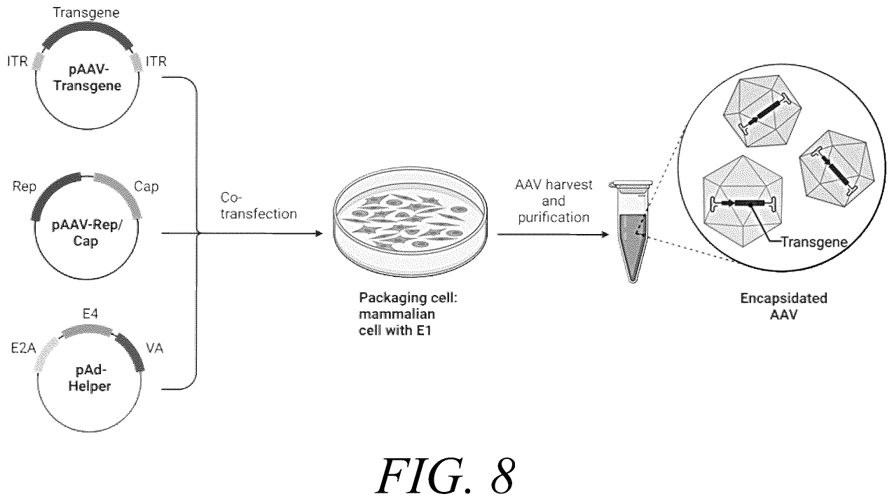

FIG. 8 is a schematic representation of production of rAAV particles by triple transfection of the E1-complementary mammalian cell line of FIG. 7 with plasmids containing genes for the replication and capsid genes of AAV, which can be from any AAV serotype, adenovirus helper genes E2A, E4, and VA RNA, which are required for AAV production, and a transgene of interest flanked by inverted terminal repeats (ITR), all of which will be packaged into a recombinant AAV particle.

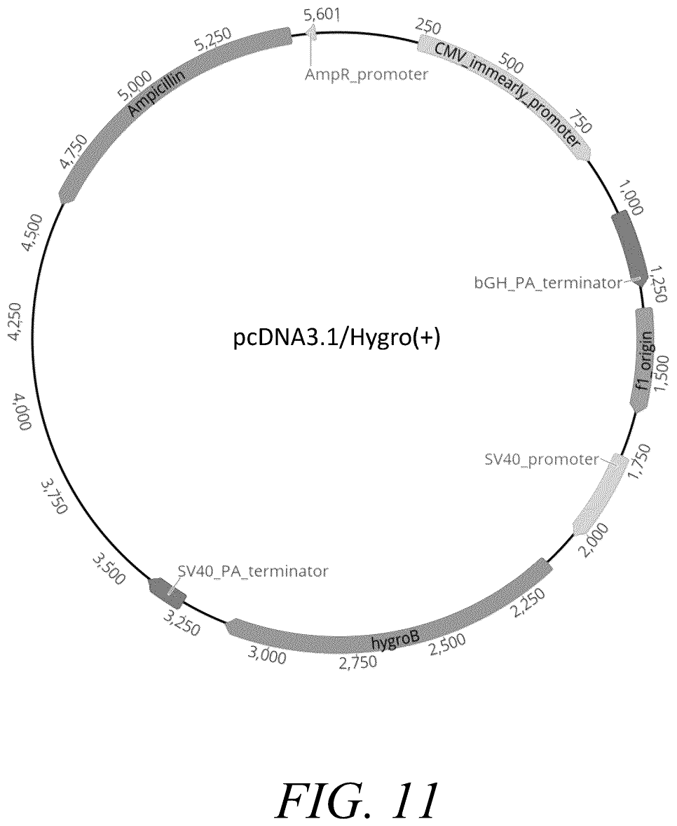

FIG. 9 is a schematic representation of a pcDNA3.1/Hygro(+) E1 WT plasmid containing an “E1 Construct” with wild-type adenovirus E1 genes E1A, E1B and IX for transformation of mammalian cells. The plasmid backbone is pcDNA3.1/Hygro(+) of FIG. 11, which includes a gene for hygromycin resistance.

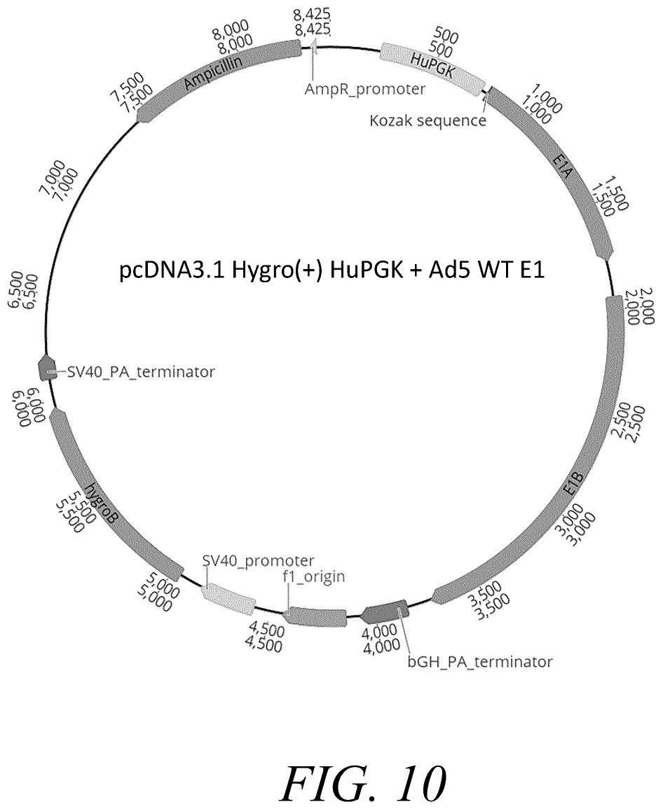

FIG. 10 is a schematic representation of a pcDNA3.1/Hygro(+) HuPGK E1A E1B bGH plasmid containing an “E1 Construct” with E1 genes E1A and E1B, a bovine growth hormone polyadenylation (bGH-poly(A)) signal and a HuPGK promoter, for transformation of mammalian cells. The plasmid backbone is pcDNA3.1/Hygro(+) of FIG. 11, which includes a gene for hygromycin resistance.

FIG. 11 is a schematic representation of a pcDNA3.1/Hygro(+) plasmid with a cloning site for insertion of portions or all of the adenovirus E1 genes (“E1 Constructs”) into the plasmid. The plasmid includes a gene for hygromycin resistance for selection of transformed mammalian cells.

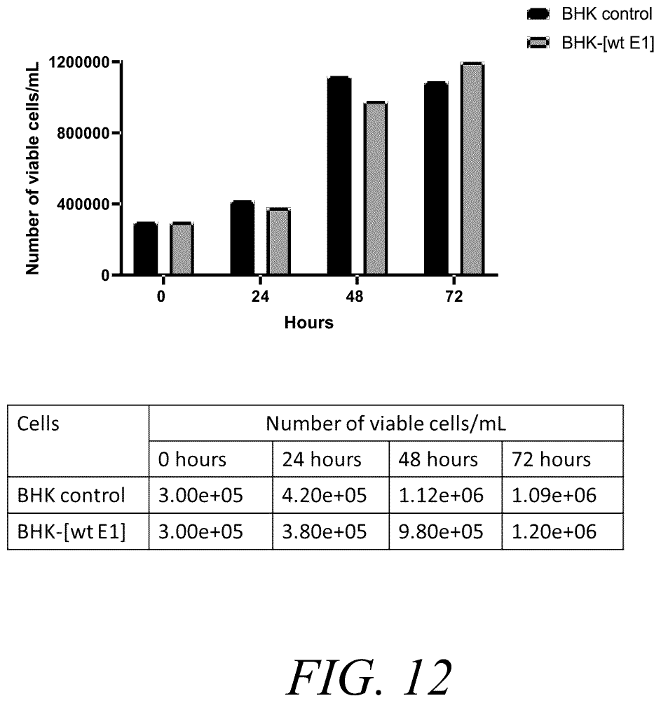

FIG. 12 is a bar graph (top panel) and table (lower panel) reporting cell viability for BHK cells transfected with the E1 WT plasmid of FIG. 9 by comparing the number of viable cells/mL for transfected and non-transfected BHK cells at time points of 0, 24, 48 and 72 hours.

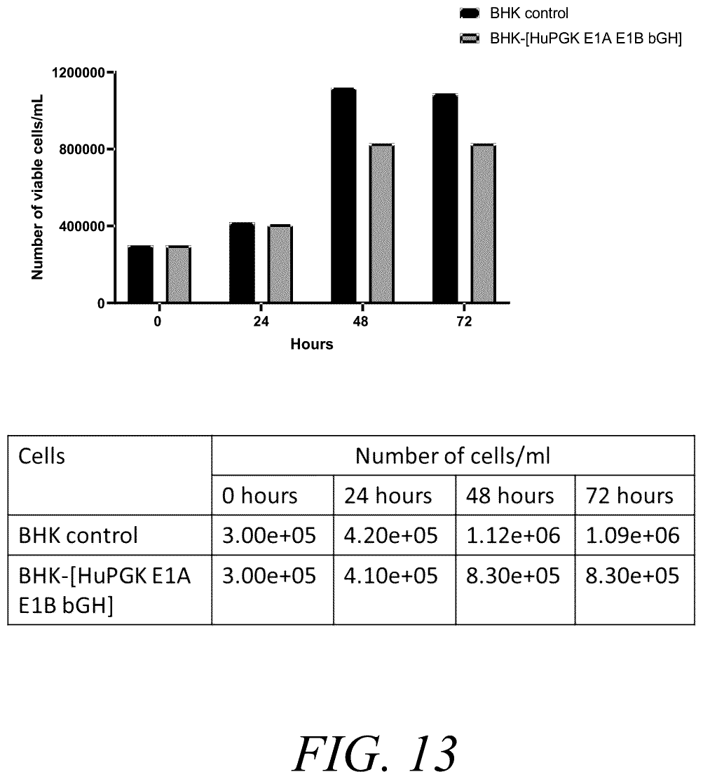

FIG. 13 is a bar graph (top panel) and table (lower panel) reporting cell viability for BHK cells transfected with the HuPGK E1A E1B bGH plasmid of FIG. 10 by comparing the number of viable cells/mL for transfected and non-transfected BHK cells at time points of 0, 24, 48 and 72 hours.

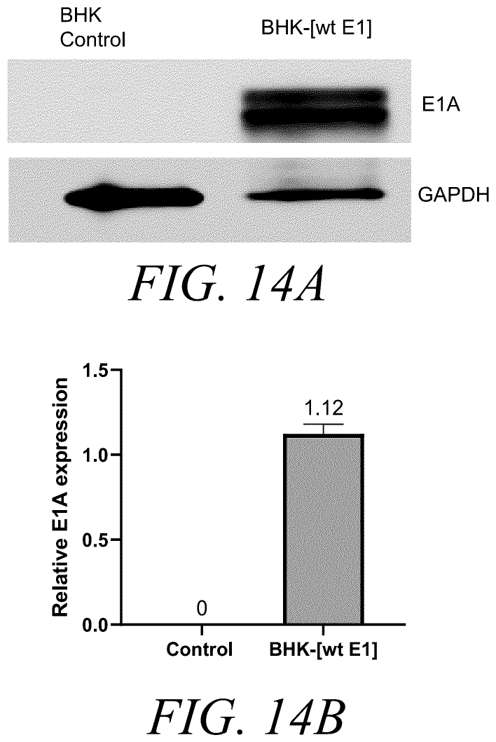

FIG. 14A is a Western blot image of E1A protein production in BHK cells transfected with the E1 WT plasmid of FIG. 9. The Western blot compares E1A protein production in transfected BHK cells with non-transfected control BHK cells. Glyceraldehyde-3-phosphate dehydrogenase (GAPDH) antibody was used as a loading control.

FIG. 14B is a bar graph representation of E1A protein production in BHK cells transfected with the E1 WT plasmid of FIG. 9. The bar graph compares and quantifies E1A protein expression in transfected BHK cells and non-transfected control BHK cells. Quantification was achieved through densitometry and error bars represent the mean±one standard deviation (SD).

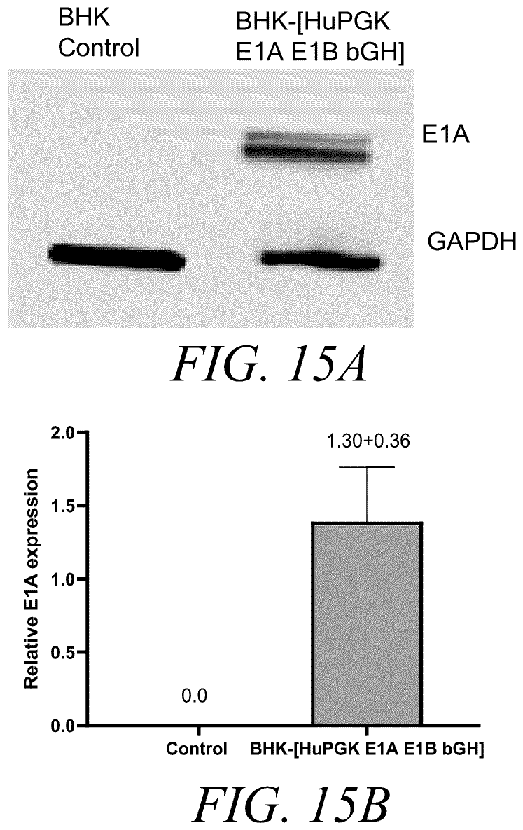

FIG. 15A is a Western blot image of E1A protein production in BHK cells transfected with the HuPGK E1A E1B bGH plasmid of FIG. 10. The Western blot compares E1A protein production in transfected BHK cells with non-transfected control BHK cells. GAPDH antibody was used as a loading control.

FIG. 15B is a bar graph representation of E1A protein production in BHK cells transfected with the HuPGK E1A E1B bGH plasmid of FIG. 10. The bar graph compares and quantifies E1A protein expression in transfected BHK cells and non-transfected control BHK cells. Quantification was achieved through densitometry and error bars represent the mean±one standard deviation (SD).

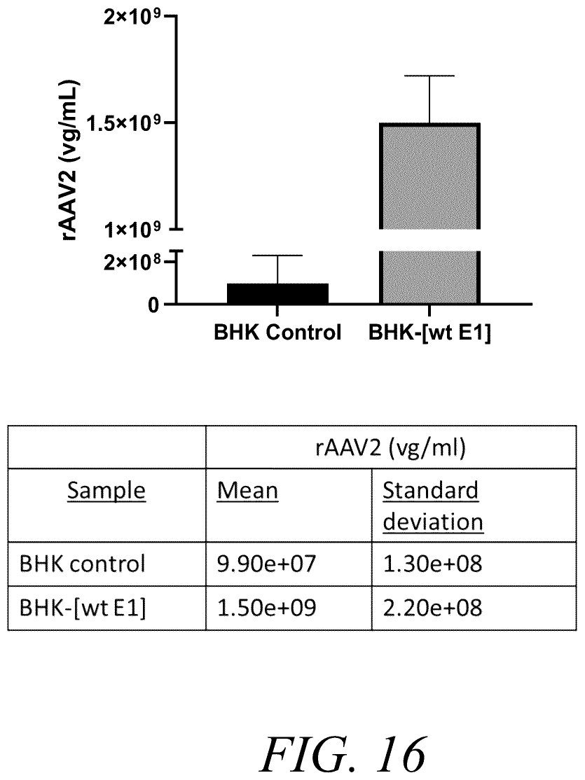

FIG. 16 is a bar graph and table comparing the production of rAAV2 particles as measured by dPCR performed using cell lysates of BHK-21 cells transformed with the plasmid of FIG. 9 containing wild-type E1 genes (E1 WT) and cell lysates of BHK-21 cells with no E1 genes (Control). Both sets of cells were triple transfected with plasmids containing AAV2 rep/cap genes, adenovirus helper genes, and the transgene green fluorescent protein. In the bar graph (top panel) and table (lower panel), rAAV production is reported as rAAV viral genomes per mL of cell culture.

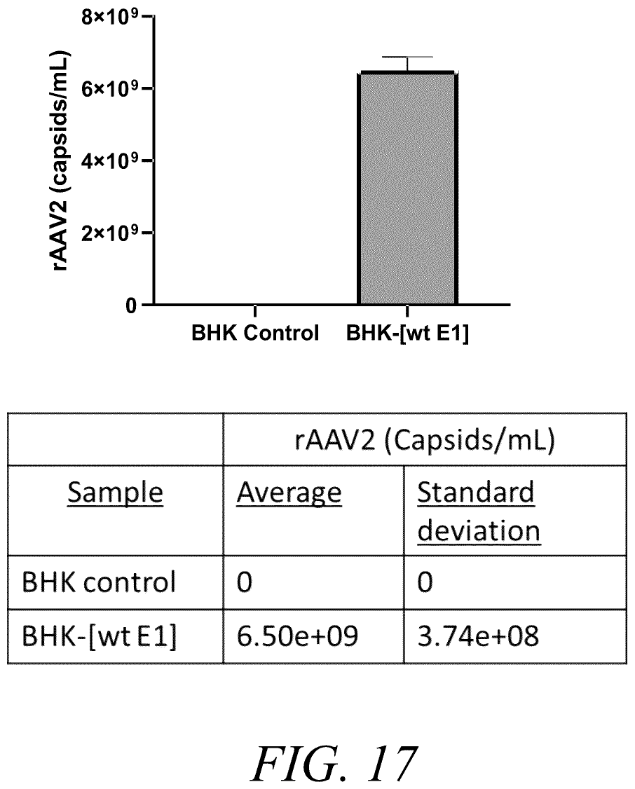

FIG. 17 is a bar graph and table comparing the production of rAAV2 capsids as measured by ELISA performed using cell lysates of BHK-21 cells transformed with the plasmid of FIG. 9 containing wild-type E1 genes (E1 WT) and cell lysates of BHK-21 cells with no E1 genes (Control). Both sets of cells were triple transfected with plasmids containing AAV2 rep/cap genes, adenovirus helper genes, and the transgene green fluorescent protein. In the bar graph (top panel) and table (lower panel), rAAV production is reported as rAAV capsids per mL of cell culture.

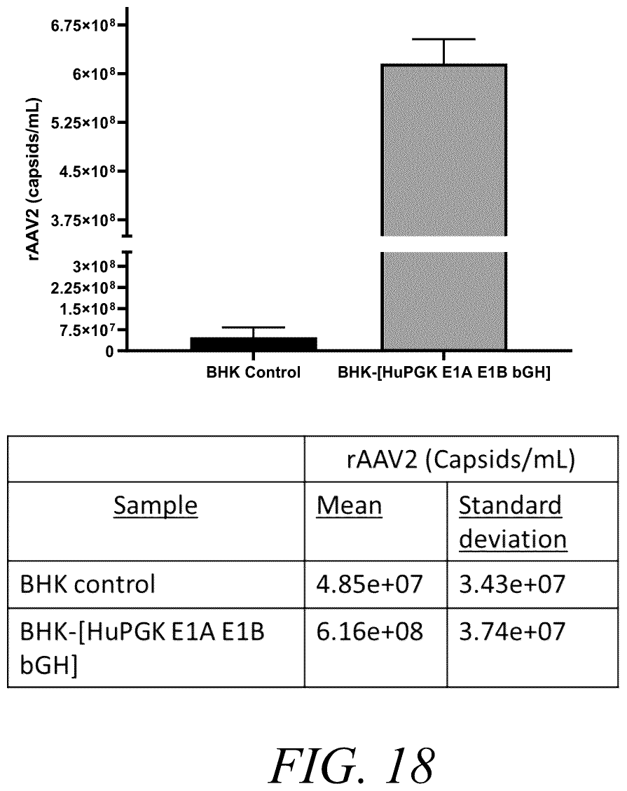

FIG. 18 is a bar graph and table comparing the production of rAAV2 capsids as measured by ELISA performed using cell lysates of BHK-21 cells transformed with the plasmid of FIG. 10 containing HuPGK E1A E1B bGH and cell lysates of BHK-21 cells with no E1 genes (Control). Both sets of cells were triple transfected with plasmids containing AAV2 rep/cap genes, adenovirus helper genes, and the transgene green fluorescent protein. The transgene was green fluorescent protein. In the bar graph (top panel) and table (lower panel), rAAV production is reported as rAAV capsids per mL of cell culture.

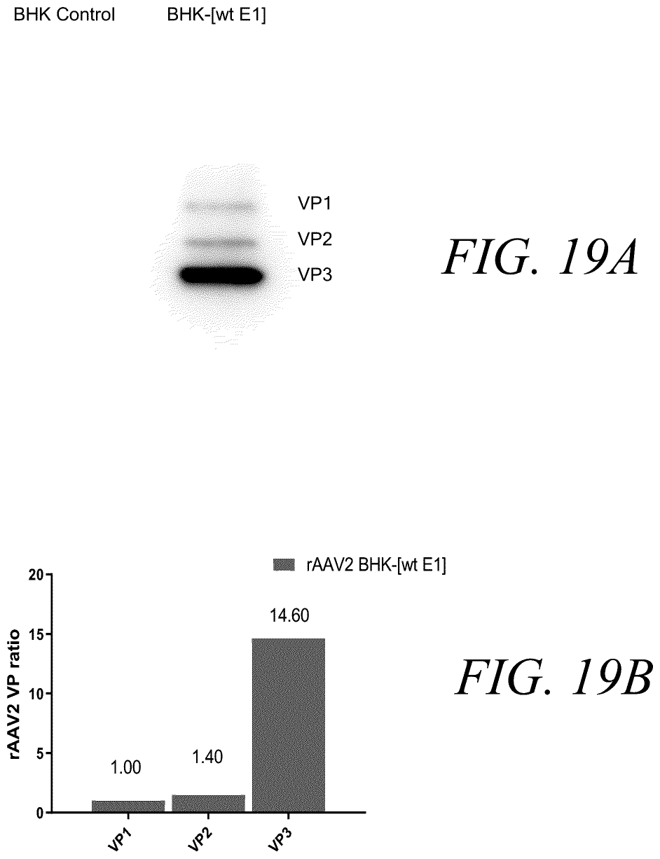

FIG. 19A is a Western blot image comparing production of rAAV2 capsid protein (VP1/VP2/VP3) in BHK cells transfected with the E1 WT plasmid of FIG. 9 and rAAV2 capsid protein production in non-transfected control BHK cells.

FIG. 19B is a bar graph identifying the ratio of VP proteins in BHK cells transfected with the E1 WT plasmid of FIG. 9 to non-transfected cells as determined using densitometry.

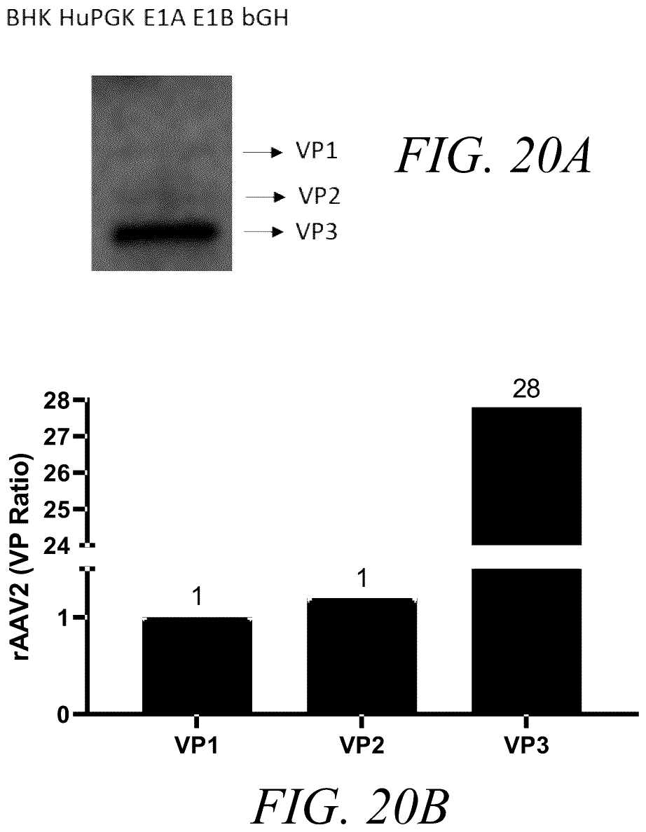

FIG. 20A is a Western blot image comparing production of rAAV2 capsid protein (VP1/VP2/VP3) in BHK cells transfected with the HuPGK E1A E1B bGH plasmid of FIG. 10 and rAAV2 capsid protein production in non-transfected control BHK cells.

FIG. 20B is a bar graph identifying the ratio of VP proteins in BHK cells transfected with the HuPGK E1A E1B bGH plasmid of FIG. 10 to non-transfected cells as determined using densitometry.

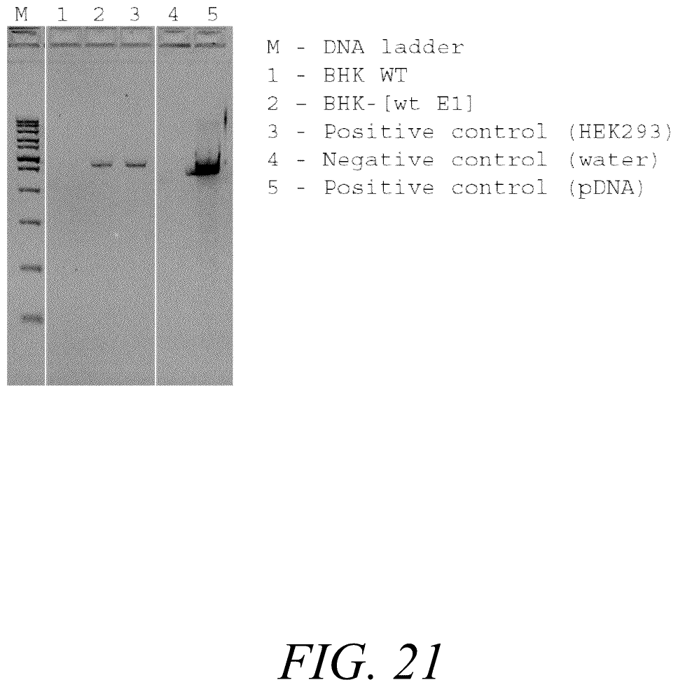

FIG. 21 is an agarose gel electrophoresis of DNA fragments produced from PCR using E1 primers of genomic DNA from BHK-21 (lane 1), genomic DNA from BHK-[wt E1] (lane 2), genomic DNA from HEK293 (lane 3), water (lane 4) and pcDNA3.1/Hygro(+) E1 WT plasmid of FIG. 9 (lane 5). Lane M is a molecular-weight DNA ladder.

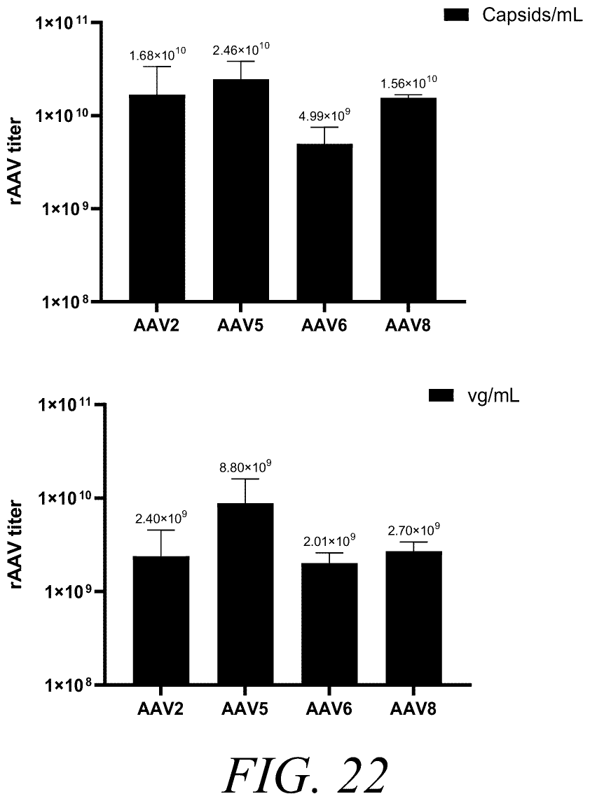

FIG. 22 is a set of bar graphs reporting rAAV production of multiple AAV serotypes in BHK-[wt E1] cells by triple transfection in serum-free media. Production of rAAV2, rAAV5, rAAV6 and rAAV8 was measured by ELISA (capsids/mL) (top panel) and dPCR (viral genomes (vg/mL)) (lower panel). The transgene was green fluorescent protein. Cells were incubated for 72 hr. post-transfection in DMEM serum-free media. Each bar represents the mean±the standard deviation from three biological replicates.

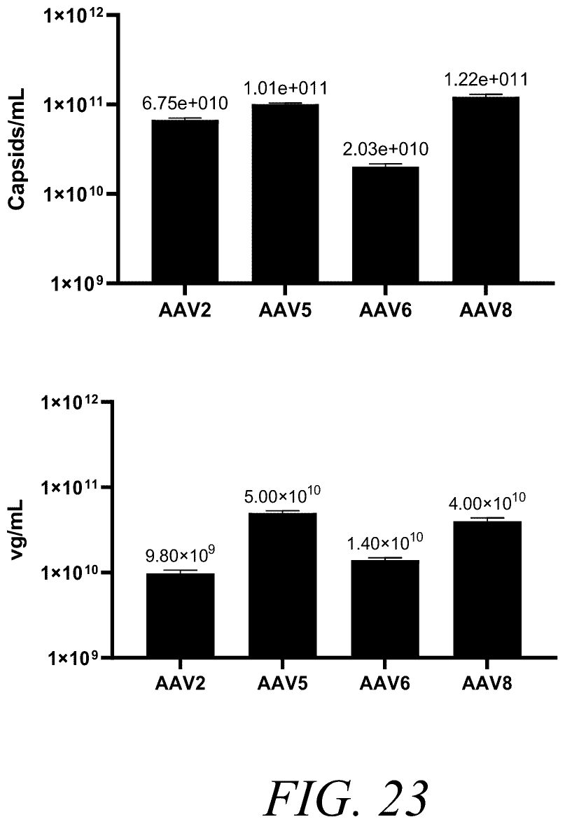

FIG. 23 is a set of bar graphs reporting rAAV production of multiple AAV serotypes in BHK-[wtE1] cells by triple transfection in reduced serum 5% FBS media. Production of rAAV2, rAAV5, rAAV6 and rAAV8 was measured by ELISA (capsids/mL) (top panel) and dPCR (viral genomes (vg/mL)) (lower panel). The transgene was green fluorescent protein. Cells were incubated for 72 hr. post-transfection in DMEM media containing 5% FBS. Each bar represents the mean±the standard deviation from technical replicates of one experiment.

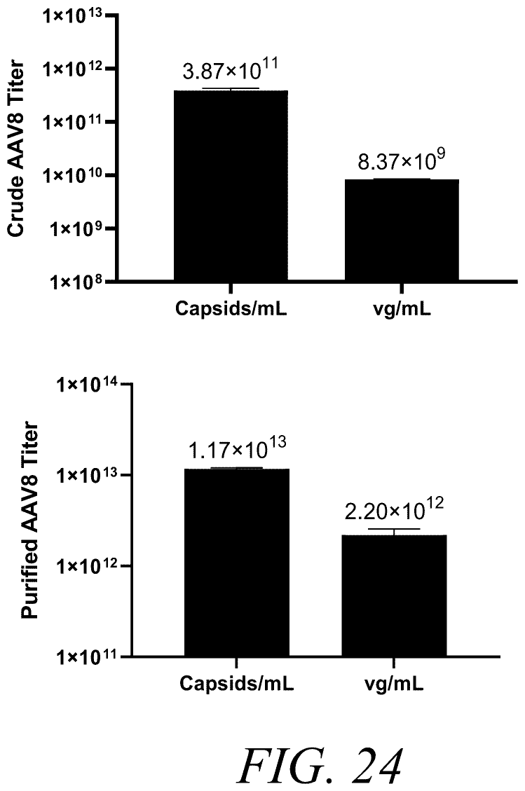

FIG. 24 contains bar graphs reporting the scaled-up production of rAAV8 in BHK-[wt E1] cells measured by ELISA (capsids/mL) and dPCR (viral genomes (vg/mL)) of rAAV8 (top panel) crude lysate and (lower panel) purified lysate. The transgene was Luciferase. Cells were incubated for 72 hr. post-transfection in DMEM media containing 5% FBS. Each bar represents the mean±the standard deviation from technical replicates of one experiment.

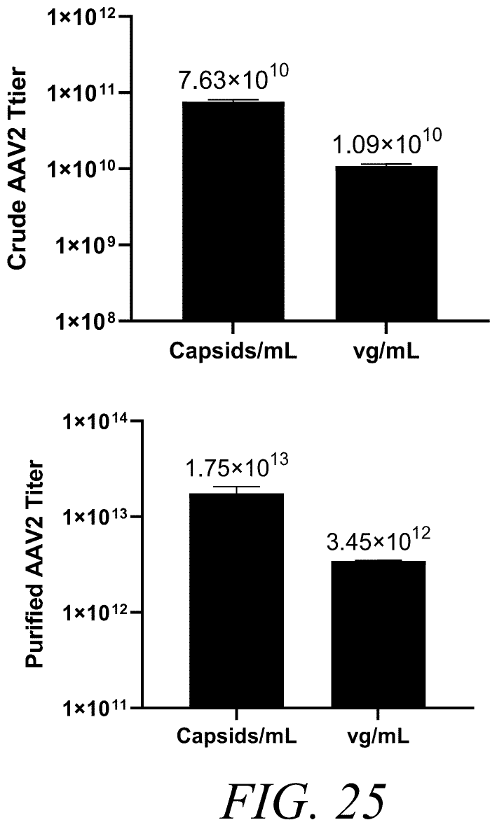

FIG. 25 contains bar graphs reporting the scaled-up production of rAAV2 in BHK-[wt E1] cells measured by ELISA (capsids/mL) and dPCR (viral genomes (vg/mL)) of rAAV2 (top panel) crude lysate and (lower panel) purified lysate. The transgene was Luciferase. Cells were incubated for 72 hr. post-transfection in DMEM media containing 5% FBS. Each bar represents the mean±the standard deviation from technical replicates of one experiment.

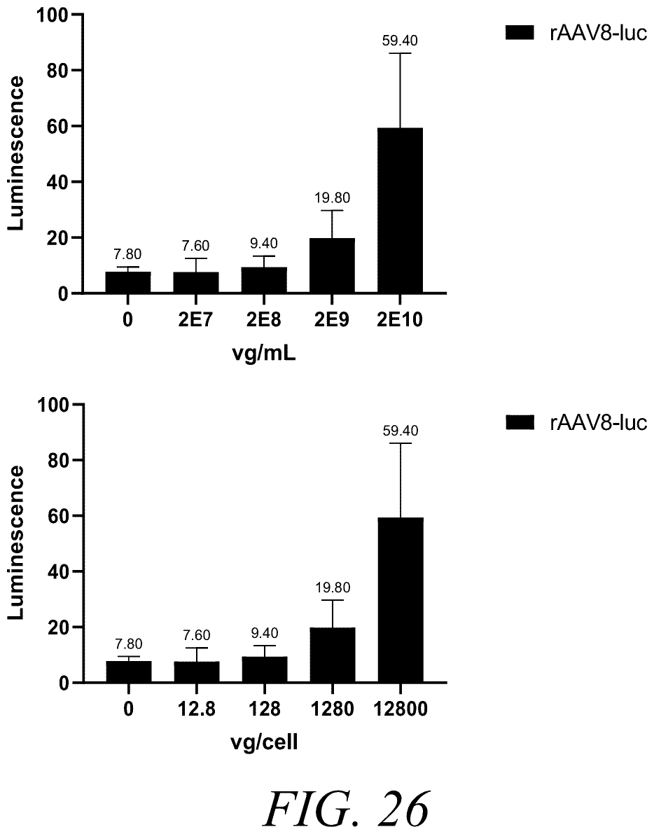

FIG. 26 contains bar graphs reporting infectivity of rAAV8-luciferase particles purified from BHK-[wt E1] cells as demonstrated by Luciferase activity from a HepG2 cell line infected with rAAV8-luciferase. Bar graphs report the luminescence of HepG2 cells infected with rAAV8-luciferase at different concentrations measured in viral genomes/mL (vg/mL) (top panel) and viral genomes/cell (vg/cell) (lower panel). Each bar represents the mean±the standard deviation from five technical replicates of one experiment.

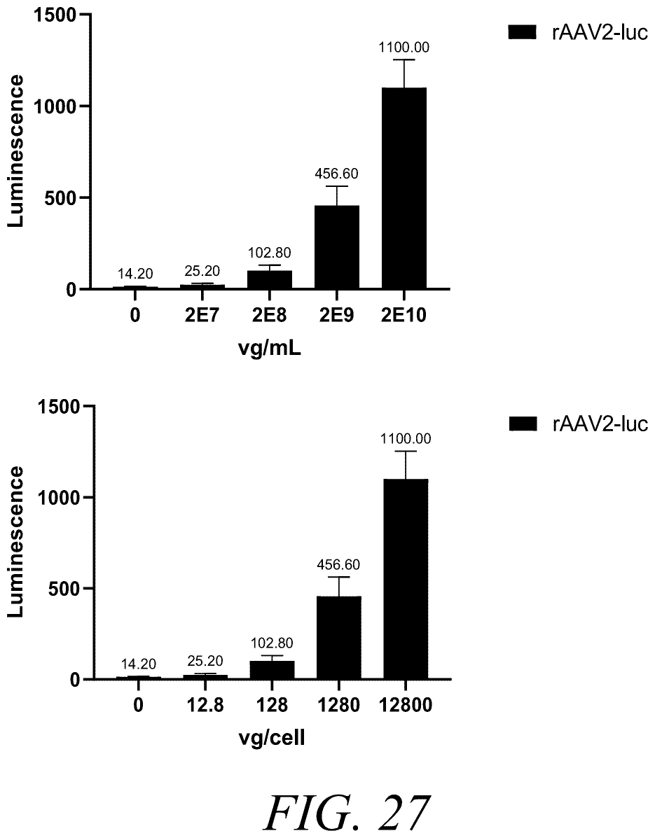

FIG. 27 contains bar graphs reporting infectivity of rAAV2-luciferase particles purified from BHK-[wt E1] cells as demonstrated by Luciferase activity from a HepG2 cell line infected with rAAV2-luciferase. Bar graphs report the luminescence of HepG2 cells infected with rAAV2-luciferase at different concentrations measured in viral genomes/mL (vg/mL) (top panel) and viral genomes/cell (vg/cell) (lower panel). Each bar represents the mean±the standard deviation from five technical replicates of one experiment.

### DETAILED DESCRIPTION OF THE INVENTION

### A. Introduction

The wild-type AAV genome contains replication and packaging, capsid, and accessory protein genes as shown in FIG. 1. The AAV lifecycle requires co-infection with a helper virus, as shown in FIG. 2. The helper virus is typically adenovirus, though other helper viruses are possible. Specific genes from adenovirus are necessary for AAV propagation, (see FIG. 3). Molecular biology techniques allow recombination of genetic elements resulting in an AAV vector that contains a transgene in place of the replication (rep) and capsid (cap) genes. The resulting recombinant AAV (rAAV) is shown in FIG. 4, with the wild-type AAV cassette in the top panel and the cassette with a promoter, transgene, and poly Adenylation sequences replacing the rep and cap genes in the lower panel.

A “vector” is a nucleic acid molecule, a plasmid, virus (e.g., AAV vector), or other vehicle that can be manipulated by insertion or incorporation of a nucleic acid. A viral vector is derived from or based upon one or more nucleic acid elements that comprise a viral genome. The term “recombinant,” as a modifier of vector, such as recombinant AAV vector, as well as a modifier of sequences such as recombinant polynucleotides and polypeptides, means that the compositions have been manipulated (i.e., engineered by recombining genetic sequences) using molecular biology techniques into a form that generally does not occur in nature. Exogenous nucleic acid is nucleic acid originating outside the organism of concern or study.

Adeno-associated virus (AAV) is a small (approximately 25 nm), non-enveloped virus of the Parvoviridae family, including twelve (12) different AAV serotypes, that infects humans and some other primate species. They are replication-deficient and in nature have linear single-stranded DNA (ssDNA) genomes. A “recombinant AAV (rAAV) vector” is derived from the wild type (wt) genome of AAV by using molecular methods to remove all or a portion the wild-type genome from the AAV genome, for example the rep/cap genes, and replacing it with a non-native nucleic acid sequence, referred to as a heterologous nucleic acid or transgene. Typically, one or both inverted terminal repeat (ITR) sequences of the AAV genome are retained and flank the cloned non-native sequence in the AAV vector, referred to as an AAV transfer plasmid.

The term “helper virus” refers to at least one of adenovirus E2A, E4 and VA RNA, or to corresponding functions of other viruses, such as herpesviruses and poxviruses, which can impart helper function to support propagation of AAV. As used herein, the term “adenovirus” refers to viruses of the family Adenoviridiae. The term “recombinant adenovirus” refers to viruses of the family Adenoviridiae capable of infecting a cell whose viral genomes have been modified through recombinant DNA techniques. The term recombinant adenovirus also includes chimeric (or even multimeric) vectors, i.e., vectors constructed using complementary coding sequences from more than one viral subtype. The term “Adenoviridae” refers collectively to adenoviruses of the genus Mastadenovirus including, but not limited to human, bovine, ovine, equine, canine, porcine, murine and simian adenovirus subgenera. In particular, human adenoviruses include the A-F subgenera as well as the individual serotypes thereof. The A-F subgenera include, but are not limited to, human adenovirus serotypes 1, 2, 3, 4, 4a, 5, 6, 7, 7a, 7d, 8, 9, 10, 11 (Ad11A and Ad11P), 12, 13, 14, 15, 16, 17, 18, 19, 20, 21, 22, 23, 24, 25, 26, 27, 28, 29, 30, 31, 32, 33, 34, 34a, 35, 35p, 36, 37, 38, 39, 40, 41, 42, 43, 44, 45, 46, 47, 48, and 91.

The adenoviral E1 gene includes E1A and E1B and refers to the early gene of the adenovirus genome that is the first gene transcribed after infection. The E1 gene referenced herein may be from human adenovirus 5 (HAdV-5), or from any other adenovirus or human adenovirus serotype. The genomic sequence of wild-type E1A is alternatively spliced into five mRNA transcripts, 9S, 10S, 11S, 12S and 13S, each coding for different non-structural proteins important for viral replication that are produced after the virus enters the host cell. The E1 gene may be modified, such as through use of different promoters, such as a human phosphoglycerate kinase promoter (HuPGK), or by inclusion of the gene encoding protein IX (pIX).

Recombinant AAV particles can be used as a pharmaceutical product by delivering a transgene that expresses a protein that provides therapeutic benefit to a patient. Production of rAAV particles requires expression of the rep, cap and helper genes and encapsulation of the transgene. As described above and shown in FIG. 5, HEK293 was created to enable production of adenoviral vectors by integrating into its genome the E1 genes (E1A and E1B), a subset of the genes required for AAV vector production. Recombinant AAV can be produced by transfection of HEK293 with plasmids containing the other necessary elements—AAV rep/cap and helper genes—as shown in FIG. 6.

Production of rAAV via triple transfection is carried out by expansion of a requisite cell line containing the complementary E1 gene from a cryopreserved stock cell bank. The three plasmids encoding the AAV rep/cap genes, helper genes and a transgene of interest flanked by the ITR sequences of AAV are added to the cells in quantities experimentally determined to provide optimal yield along with a transfection reagent. There are several options for transfection, including calcium phosphate precipitation and use of liposomes like polyethylenimine. Transfected cells are grown in a suitable media for an appropriate time. The cells are harvested and lysed and the supernatant is separated and collected from the cell debris. Recombinant AAV particles are purified from the supernatant using either density gradient ultracentrifugation or chromatography, or other means of purification known in the art. The purified rAAV particles are concentrated and formulated in an appropriate buffer with components to reduce degradation and loss through aggregation or adherence to the vessel or transfer device. The rAAV particles can transduce, either ex vivo or in vivo, an appropriate animal cell resulting in expression of the transgene.

### B. Sequences

Table 1 below provides examples of the nucleotide sequences of human adenovirus serotype 5 E1 and plasmids containing all or part of the E1 gene region.

TABLE 1
Sequences of the Invention

| Sequence Name | Sequence Identifier |
|---|---|
| Nucleotide sequence encoding human | SEQ ID NO: 1 |
| adenovirus type 5, E1 CDS, wild type | |
| Nucleotide sequence for E1A and E1B CDS | SEQ ID NO: 2 |
| with bGH and HuPGK promoter | |
| Vector pcDNA3.1/Hygro(+) | SEQ ID NO: 3 |
| Vector pcDNA3.1/Hygro(+) WT E1 | SEQ ID NO: 4 |
| Vector pcDNA3.1/Hygro(+) HuPGK E1A E1B bGH | SEQ ID NO: 5 |

The nucleotide sequence encoding human adenovirus type 5, E1 CDS, wild type (SEQ ID NO: 1) is displayed in Table 2, below.

TABLE 2
Sequence encoding human adenovirus type 5, E1 CDS, wild type

| | | | | | | |
|---|---|---|---|---|---|---|
| 1 | catcatcaat | aatatacctt | attttggatt | gaagccaata | tgataatgag | ggggtggagt |
| 61 | ttgtgacgtg | gcgcggggcg | tgggaacggg | gcgggtgacg | tagtagtgtg | gcggaagtgt |
| 121 | gatgttgcaa | gtgtggcgga | acacatgtaa | gcgacggatg | tggcaaaagt | gacgtttttg |
| 181 | gtgtgcgccg | gtgtacacag | gaagtgacaa | ttttcgcgcg | gttttaggcg | gatgttgtag |
| 241 | taaatttggg | cgtaaccgag | taagatttgg | ccattttcgc | gggaaaactg | aataagagga |
| 301 | agtgaaatct | gaataatttt | gtgttactca | tagcgcgtaa | tatttgtcta | gggccgcggg |
| 361 | gactttgacc | gtttacgtgg | agactcgccc | aggtgttttt | ctcaggtgtt | ttccgcgttc |
| 421 | cgggtcaaag | ttggcgtttt | attattatag | tcagctgacg | tgtagtgtat | ttatacccgg |
| 481 | tgagttcctc | aagaggccac | tcttgagtgc | cagcgagtag | agttttctcc | tccgagccgc |
| 541 | tccgacaccg | ggactgaaaa | tgagacatat | tatctgccac | ggaggtgtta | ttaccgaaga |
| 601 | aatggccgcc | agtcttttgg | accagctgat | cgaagaggta | ctggctgata | atcttccacc |
| 661 | tcctagccat | tttgaaccac | ctacccttca | cgaactgtat | gatttagacg | tgacggcccc |
| 721 | cgaagatccc | aacgaggagg | cggtttcgca | gatttttccc | gactctgtaa | tgttggcggt |
| 781 | gcaggaaggg | attgacttac | tcacttttcc | gccggcgccc | ggttctccgg | agccgcctca |
| 841 | cctttcccgg | cagcccgagc | agccggagca | gagagccttg | ggtccggttt | ctatgccaaa |
| 901 | ccttgtaccg | gaggtgatcg | atcttacctg | ccacgaggct | ggctttccac | ccagtgacga |
| 961 | cgaggatgaa | gagggtgagg | agtttgtgtt | agattatgtg | gagcaccccg | ggcacggttg |
| 1021 | caggtcttgt | cattatcacc | ggaggaatac | gggggaccca | gatattatgt | gttcgctttg |
| 1081 | ctatatgagg | acctgtggca | tgtttgtcta | cagtaagtga | aaattatggg | cagtgggtga |
| 1141 | tagagtggtg | ggtttggtgt | ggtaattttt | tttttaattt | ttacagtttt | gtggtttaaa |
| 1201 | gaattttgta | ttgtgatttt | tttaaaaggt | cctgtgtctg | aacctgagcc | tgagcccgag |
| 1261 | ccagaaccgg | agcctgcaag | acctacccgc | cgtcctaaaa | tggcgcctgc | tatcctgaga |
| 1321 | cgcccgacat | cacctgtgtc | tagagaatgc | aatagtagta | cggatagctg | tgactccggt |
| 1381 | ccttctaaca | cacctcctga | gatacacccg | gtggtcccgc | tgtgccccat | taaaccagtt |
| 1441 | gccgtgagag | ttggtgggcg | tcgccaggct | gtggaatgta | tcgaggactt | gcttaacgag |
| 1501 | cctgggcaac | ctttggactt | gagctgtaaa | cgccccaggc | cataaggtgt | aaacctgtga |
| 1561 | ttgcgtgtgt | ggttaacgcc | tttgtttgct | gaatgagttg | atgtaagttt | aataaagggt |
| 1621 | gagataatgt | ttaacttgca | tggcgtgtta | aatggggcgg | ggcttaaagg | gtatataatg |
| 1681 | cgccgtgggc | taatcttggt | tacatctgac | ctcatggagg | cttgggagtg | tttggaagat |
| 1741 | ttttctgctg | tgcgtaactt | gctggaacag | agctctaaca | gtacctcttg | gttttggagg |
| 1801 | tttctgtggg | gctcatccca | ggcaaagtta | gtctgcagaa | ttaaggagga | ttacaagtgg |
| 1861 | gaatttgaag | agcttttgaa | atcctgtggt | gagctgtttg | attctttgaa | tctgggtcac |
| 1921 | caggcgcttt | tccaagagaa | ggtcatcaag | actttggatt | tttccacacc | ggggcgcgct |
| 1981 | gcggctgctg | ttgctttttt | gagttttata | aaggataaat | ggagcgaaga | aacccatctg |
| 2041 | agcggggggt | acctgctgga | ttttctggcc | atgcatctgt | ggagagcggt | tgtgagacac |
| 2101 | aagaatcgcc | tgctactgtt | gtcttccgtc | cgcccggcga | taataccgac | ggaggagcag |
| 2161 | cagcagcagc | aggaggaagc | caggcggcgg | cggcaggagc | agagcccatg | gaacccgaga |
| 2221 | gccggcctgg | accctcggga | atgaatgttg | tacaggtggc | tgaactgtat | ccagaactga |
| 2281 | gacgcatttt | gacaattaca | gaggatgggc | aggggctaaa | gggggtaaag | agggagcggg |
| 2341 | gggcttgtga | ggctacagag | gaggctagga | atctagcttt | tagcttaatg | accagacacc |
| 2401 | gtcctgagtg | tattactttt | caacagatca | aggataattg | cgctaatgag | cttgatctgc |
| 2461 | tggcgcagaa | gtattccata | gagcagctga | ccacttactg | gctgcagcca | ggggatgatt |
| 2521 | ttgaggaggc | tattagggta | tatgcaaagg | tggcacttag | gccagattgc | aagtacaaga |
| 2581 | tcagcaaact | tgtaaatatc | aggaattgtt | gctacatttc | tgggaacggg | gccgaggtgg |
| 2641 | agatagatac | ggaggatagg | gtggccttta | gatgtagcat | gataaatatg | tggccggggg |
| 2701 | tgcttggcat | ggacggggtg | gttattatga | atgtaaggtt | tactggcccc | aattttagcg |
| 2761 | gtacggtttt | cctggccaat | accaacctta | tcctacacgg | tgtaagcttc | tatgggttta |
| 2821 | acaatacctg | tgtggaagcc | tggaccgatg | taagggttcg | gggctgtgcc | ttttactgct |
| 2881 | gctggaaggg | ggtggtgtgt | cgccccaaaa | gcagggcttc | aattaagaaa | tgcctctttg |
| 2941 | aaaggtgtac | cttgggtatc | ctgtctgagg | gtaactccag | ggtgcgccac | aatgtggcct |
| 3001 | ccgactgtgg | ttgcttcatg | ctagtgaaaa | gcgtggctgt | gattaagcat | aacatggtat |
| 3061 | gtggcaactg | cgaggacagg | gcctctcaga | tgctgacctg | ctcggacggc | aactgtcacc |
| 3121 | tgctgaagac | cattcacgta | gccagccact | ctcgcaaggc | ctggccagtg | tttgagcata |
| 3181 | acatactgac | ccgctgttcc | ttgcatttgg | gtaacaggag | gggggtgttc | ctaccttacc |
| 3241 | aatgcaattt | gagtcacact | aagatattgc | ttgagcccga | gagcatgtcc | aaggtgaacc |
| 3301 | tgaacggggt | gtttgacatg | accatgaaga | tctggaaggt | gctgaggtac | gatgagaccc |
| 3361 | gcaccaggtg | cagaccctgc | gagtgtggcg | gtaaacatat | taggaaccag | cctgtgatgc |
| 3421 | tggatgtgac | cgaggagctg | aggcccgatc | acttggtgct | ggcctgcacc | cgcgctgagt |
| 3481 | ttggctctag | cgatgaagat | acagattgag | gtactgaaat | gtgtgggcgt | ggcttaaggg |
| 3541 | tgggaaagaa | tatataaggt | gggggtctta | tgtagttttg | tatctgtttt | gcagcagccg |
| 3601 | ccgccgccat | gagcaccaac | tcgtttgatg | gaagcattgt | gagctcatat | ttgacaacgc |
| 3661 | gcatgccccc | atgggccggg | gtgcgtcaga | atgtgatggg | ctccagcatt | gatggtcgcc |
| 3721 | ccgtcctgcc | cgcaaactct | actaccttga | cctacgagac | cgtgtctgga | acgccgttgg |
| 3781 | agactgcagc | ctccgccgcc | gcttcagccg | ctgcagccac | cgcccgcggg | attgtgactg |
| 3841 | actttgcttt | cctgagcccg | cttgcaagca | gtgcagcttc | ccgttcatcc | gcccgcgatg |
| 3901 | acaagttgac | ggctcttttg | gcacaattgg | attctttgac | ccgggaactt | aatgtcgttt |
| 3961 | ctcagcagct | gttggatctg | cgccagcagg | tttctgccct | gaaggcttcc | tcccctccca |
| 4021 | atgcggttta | aaacataaat | aaaaaaccag | actctgtttg | gatttggatc | aagcaagtgt |
| 4081 | cttgctgtct | ttatttaggg | gttttgcgcg | cgcggtaggc | ccgggaccag | cggtctcggt |
| 4141 | cgttgagggt | cctgtgtatt | ttttccagga | cgtggtaaag | gtgactctgg | atgttcagat |
| 4201 | acatgggcat | aagcccgtct | ctggggtgga | ggtagcacca | ctgcagagct | tcatgctgcg |
| 4261 | gggtggtgtt | gtagatgatc | cagtcgtagc | aggagcgctg | ggcgtggtgc | ctaaaaatgt |
| 4321 | ctttcagtag | caagctgatt | gcca | | | |

The nucleotide sequence encoding E1A and E1B CDS with bGH and HuPGK promoter (SEQ ID NO: 2) is displayed in Table 3, below.

TABLE 3
Sequence encoding E1A and E1B CDS with bGH and HuPGK promoter

| | | | | | | |
|---|---|---|---|---|---|---|
| 1 | ggggttgggg | ttgcgccttt | tccaaggcag | ccctgggttt | gcgcagggac | gcggctgctc |
| 61 | tgggcgtggt | tccgggaaac | gcagcggcgc | cgaccctggg | tctcgcacat | tcttcacgtc |
| 121 | cgttcgcagc | gtcacccgga | tcttcgccgc | tacccttgtg | ggccccccgg | cgacgcttcc |
| 181 | tgctccgccc | ctaagtcggg | aaggttcctt | gcggttcgcg | gcgtgccgga | cgtgacaaac |
| 241 | ggaagccgca | cgtctcacta | gtaccctcgc | agacggacag | cgccagggag | caatggcagc |
| 301 | gcgccgaccg | cgatgggctg | tggccaatag | cggctgctca | gcagggcgcg | ccgagagcag |
| 361 | cggccgggaa | ggggcggtgc | gggaggcggg | gtgtggggcg | gtagtgtggg | ccctgttcct |
| 421 | gcccgcgcgg | tgttccgcat | tctgcaagcc | tccggagcgc | acgtcggcag | tcggctccct |
| 481 | cgttgaccga | atcaccgacc | tctctcccca | gccgggtacg | tcgctagagg | atcgaaccct |
| 541 | tgccaccatg | agacatatta | tctgccacgg | aggtgttatt | accgaagaaa | tggccgccag |
| 601 | tcttttggac | cagctgatcg | aagaggtact | ggctgataat | cttccacctc | ctagccattt |
| 661 | tgaaccacct | acccttcacg | aactgtatga | tttagacgtg | acggcccccg | aagatcccaa |
| 721 | cgaggaggcg | gtttcgcaga | tttttcccga | ctctgtaatg | ttggcggtgc | aggaagggat |
| 781 | tgacttactc | acttttccgc | cggcgcccgg | ttctccggag | ccgcctcacc | tttcccggca |
| 841 | gcccgagcag | ccggagcaga | gagccttggg | tccggtttct | atgccaaacc | ttgtaccgga |
| 901 | ggtgatcgat | cttacctgcc | acgaggctgg | ctttccaccc | agtgacgacg | aggatgaaga |
| 961 | gggtgaggag | tttgtgttag | attatgtgga | gcaccccggg | cacggttgca | ggtcttgtca |
| 1021 | ttatcaccgg | aggaatacgg | gggacccaga | tattatgtgt | tcgctttgct | atatgaggac |
| 1081 | ctgtggcatg | tttgtctaca | gtaagtgaaa | attatgggca | gtgggtgata | gagtggtggg |
| 1141 | tttggtgtgg | taattttttt | tttaattttt | acagttttgt | ggtttaaaga | attttgtatt |
| 1201 | gtgatttttt | taaaaggtcc | tgtgtctgaa | cctgagcctg | agcccgagcc | agaaccggag |
| 1261 | cctgcaagac | ctacccgccg | tcctaaaatg | gcgcctgcta | tcctgagacg | cccgacatca |
| 1321 | cctgtgtcta | gagaatgcaa | tagtagtacg | gatagctgtg | actccggtcc | ttctaacaca |
| 1381 | cctcctgaga | tacacccggt | ggtcccgctg | tgccccatta | aaccagttgc | cgtgagagtt |
| 1441 | ggtgggcgtc | gccaggctgt | ggaatgtatc | gaggacttgc | ttaacgagcc | tgggcaacct |
| 1501 | ttggacttga | gctgtaaacg | ccccaggcca | taaggtgtaa | acctgtgatt | gcgtgtgtgg |
| 1561 | ttaacgcctt | tgtttgctga | atgagttgat | gtaagtttaa | taaagggtga | gataatgttt |
| 1621 | aacttgcatg | gcgtgttaaa | tggggcgggg | cttaaagggt | atataatgcg | ccgtgggcta |
| 1681 | atcttggtta | catctgacct | catggaggct | tgggagtgtt | tggaagattt | ttctgctgtg |
| 1741 | cgtaacttgc | tggaacagag | ctctaacagt | acctcttggt | tttggaggtt | tctgtggggc |
| 1801 | tcatcccagg | caaagttagt | ctgcagaatt | aaggaggatt | acaagtggga | atttgaagag |
| 1861 | cttttgaaat | cctgtggtga | gctgtttgat | tctttgaatc | tgggtcacca | ggcgcttttc |
| 1921 | caagagaagg | tcatcaagac | tttggatttt | tccacaccgg | ggcgcgctgc | ggctgctgtt |
| 1981 | gcttttttga | gttttataaa | ggataaatgg | agcgaagaaa | cccatctgag | cggggggtac |
| 2041 | ctgctggatt | ttctggccat | gcatctgtgg | agagcggttg | tgagacacaa | gaatcgcctg |
| 2101 | ctactgttgt | cttccgtccg | cccggcgata | ataccgacgg | aggagcagca | gcagcagcag |
| 2161 | gaggaagcca | ggcggcggcg | gcaggagcag | agcccatgga | acccgagagc | cggcctggac |
| 2221 | cctcgggaat | gaatgttgta | caggtggctg | aactgtatcc | agaactgaga | cgcattttga |
| 2281 | caattacaga | ggatgggcag | gggctaaagg | gggtaaagag | ggagcggggg | gcttgtgagg |
| 2341 | ctacagagga | ggctaggaat | ctagctttta | gcttaatgac | cagacaccgt | cctgagtgta |
| 2401 | ttacttttca | acagatcaag | gataattgcg | ctaatgagct | tgatctgctg | gcgcagaagt |
| 2461 | attccataga | gcagctgacc | acttactggc | tgcagccagg | ggatgatttt | gaggaggcta |
| 2521 | ttagggtata | tgcaaaggtg | gcacttaggc | cagattgcaa | gtacaagatc | agcaaacttg |
| 2581 | taaatatcag | gaattgttgc | tacatttctg | ggaacggggc | cgaggtggag | atagatacgg |
| 2641 | aggatagggt | ggcctttaga | tgtagcatga | taaatatgtg | gccgggggtg | cttggcatgg |
| 2701 | acggggtggt | tattatgaat | gtaaggttta | ctggccccaa | ttttagcggt | acggttttcc |
| 2761 | tggccaatac | caaccttatc | ctacacggtg | taagcttcta | tgggtttaac | aatacctgtg |
| 2821 | tggaagcctg | gaccgatgta | agggttcggg | gctgtgcctt | ttactgctgc | tggaaggggg |
| 2881 | tggtgtgtcg | ccccaaaagc | agggcttcaa | ttaagaaatg | cctctttgaa | aggtgtacct |
| 2941 | tgggtatcct | gtctgagggt | aactccaggg | tgcgccacaa | tgtggcctcc | gactgtggtt |
| 3001 | gcttcatgct | agtgaaaagc | gtggctgtga | ttaagcataa | catggtatgt | ggcaactgcg |
| 3061 | aggacagggc | ctctcagatg | ctgacctgct | cggacggcaa | ctgtcacctg | ctgaagacca |
| 3121 | ttcacgtagc | cagccactct | cgcaaggcct | ggccagtgtt | tgagcataac | atactgaccc |
| 3181 | gctgttcctt | gcatttgggt | aacaggaggg | gggtgttcct | accttaccaa | tgcaatttga |
| 3241 | gtcacactaa | gatattgctt | gagcccgaga | gcatgtccaa | ggtgaacctg | aacggggtgt |
| 3301 | ttgacatgac | catgaagatc | tggaaggtgc | tgaggtacga | tgagacccgc | accaggtgca |
| 3361 | gaccctgcga | gtgtggcggt | aaacatatta | ggaaccagcc | tgtgatgctg | gatgtgaccg |
| 3421 | aggagctgag | gcccgatcac | ttggtgctgg | cctgcacccg | cgctgagttt | ggctctagcg |
| 3481 | atgaagatac | agattgaaag | cttggtaccg | agctcggatc | cactagtcca | gtgtggtgga |
| 3541 | attctgcaga | tatccagcac | agtggcggcc | gctcgagtct | agagggcccg | tttaaacccg |
| 3601 | ctgatcagcc | tcgactgtgc | cttctagttg | ccagccatct | gttgtttgcc | cctcccccgt |
| 3661 | gccttccttg | accctggaag | gtgccactcc | cactgtcctt | tcctaataaa | atgaggaaat |
| 3721 | tgcatcgcat | tgtctgagta | ggtgtcattc | tattctgggg | ggtggggtgg | ggcaggacag |
| 3781 | caagggggag | gattgggaag | acaatagcag | gcatgctggg | gatgcggtgg | gctctatgg |

The nucleotide sequence for vector pcDNA3.1/Hygro(+) (SEQ ID NO: 3) is displayed in Table 4, below.

TABLE 4
Vector pcDNA3.1/Hygro(+)

| | | | | | | |
|---|---|---|---|---|---|---|
| 1 | gacggatcgg | gagatctccc | gatcccctat | ggtcgactct | cagtacaatc | tgctctgatg |
| 61 | ccgcatagtt | aagccagtat | ctgctccctg | cttgtgtgtt | ggaggtcgct | gagtagtgcg |
| 121 | cgagcaaaat | ttaagctaca | acaaggcaag | gcttgaccga | caattgcatg | aagaatctgc |
| 181 | ttagggttag | gcgttttgcg | ctgcttcgcg | atgtacgggc | cagatatacg | cgttgacatt |
| 241 | gattattgac | tagttattaa | tagtaatcaa | ttacggggtc | attagttcat | agcccatata |
| 301 | tggagttccg | cgttacataa | cttacggtaa | atggcccgcc | tggctgaccg | cccaacgacc |
| 361 | cccgcccatt | gacgtcaata | atgacgtatg | ttcccatagt | aacgccaata | gggactttcc |
| 421 | attgacgtca | atgggtggac | tatttacggt | aaactgccca | cttggcagta | catcaagtgt |
| 481 | atcatatgcc | aagtacgccc | cctattgacg | tcaatgacgg | taaatggccc | gcctggcatt |
| 541 | atgcccagta | catgacctta | tgggactttc | ctacttggca | gtacatctac | gtattagtca |
| 601 | tcgctattac | catggtgatg | cggttttggc | agtacatcaa | tgggcgtgga | tagcggtttg |
| 661 | actcacgggg | atttccaagt | ctccacccca | ttgacgtcaa | tgggagttt | ttttggcacc |
| 721 | aaaatcaacg | ggactttcca | aaatgtcgta | acaactccgc | cccattgacg | caaatgggcg |
| 781 | gtaggcgtgt | acggtgggag | gtctatataa | gcagagctct | ctggctaact | agagaaccca |
| 841 | ctgcttactg | gcttatcgaa | attaatacga | ctcactatag | ggagacccaa | gctggctagc |
| 901 | gtttaaactt | aagcttggta | ccgagctcgg | atccactagt | ccagtgtggt | ggaattctgc |
| 961 | agatatccag | cacagtggcg | gccgctcgag | tctagagggc | ccgtttaaac | ccgctgatca |
| 1021 | gcctcgactg | tgccttctag | ttgccagcca | tctgttgttt | gcccctcccc | cgtgccttcc |
| 1081 | ttgaccctgg | aaggtgccac | tcccactgtc | ctttcctaat | aaaatgagga | aattgcatcg |
| 1141 | cattgtctga | gtaggtgtca | ttctattctg | gggggtgggg | tggggcagga | cagcaagggg |
| 1201 | gaggattggg | aagacaatag | caggcatgct | ggggatgcgg | tgggctctat | ggcttctgag |
| 1261 | gcggaaagaa | ccagctgggg | ctctaggggg | tatccccacg | cgccctgtag | cggcgcatta |
| 1321 | agcgcggcgg | gtgtggtggt | tacgcgcagc | gtgaccgcta | cacttgccag | cgccctagcg |
| 1381 | cccgctcctt | tcgctttctt | cccttccttt | ctcgccacgt | tcgccggctt | tccccgtcaa |
| 1441 | gctctaaatc | ggggcatccc | tttagggttc | cgatttagtg | ctttacggca | cctcgacccc |
| 1501 | aaaaaacttg | attagggtga | tggttcacgt | agtgggccat | cgccctgata | gacggttttt |
| 1561 | cgccctttga | cgttggagtc | cacgttcttt | aatagtggac | tcttgttcca | aactggaaca |
| 1621 | acactcaacc | ctatctcggt | ctattctttt | gatttataag | ggattttggg | gatttcggcc |
| 1681 | tattggttaa | aaaatgagct | gatttaacaa | aaatttaacg | cgaattaatt | ctgtggaatg |
| 1741 | tgtgtcagtt | agggtgtgga | aagtccccag | gctccccagg | caggcagaag | tatgcaaagc |
| 1801 | atgcatctca | attagtcagc | aaccaggtgt | ggaaagtccc | caggctcccc | agcaggcaga |
| 1861 | agtatgcaaa | gcatgcatct | caattagtca | gcaaccatag | tcccgcccct | aactccgccc |
| 1921 | atcccgcccc | taactccgcc | cagttccgcc | cattctccgc | cccatggctg | actaattttt |
| 1981 | tttatttatg | cagaggccga | ggccgcctct | gcctctgagc | tattccagaa | gtagtgagga |
| 2041 | ggcttttttg | gaggcctagg | cttttgcaaa | aagctcccgg | gagcttgtat | atccattttc |
| 2101 | ggatctgatc | agcacgtgat | gaaaaagcct | gaactcaccg | cgacgtctgt | cgagaagttt |
| 2161 | ctgatcgaaa | agttcgacag | cgtctccgac | ctgatgcagc | tctcggaggg | cgaagaatct |
| 2221 | cgtgctttca | gcttcgatgt | aggagggcgt | ggatatgtcc | tgcgggtaaa | tagctgcgcc |
| 2281 | gatggtttct | acaaagatcg | ttatgtttat | cggcactttg | catcggccgc | gctcccgatt |
| 2341 | ccggaagtgc | ttgacattgg | ggaattcagc | gagagcctga | cctattgcat | ctcccgccgt |
| 2401 | gcacagggtg | tcacgttgca | agacctgcct | gaaaccgaac | tgcccgctgt | tctgcagccg |
| 2461 | gtcgcggagg | ccatggatgc | gatcgctgcg | gccgatctta | gccagacgag | cgggttcggc |
| 2521 | ccattcggac | cgcaaggaat | cggtcaatac | actacatggc | gtgatttcat | atgcgcgatt |
| 2581 | gctgatcccc | atgtgtatca | ctggcaaact | gtgatggacg | acaccgtcag | tgcgtccgtc |
| 2641 | gcgcaggctc | tcgatgagct | gatgctttgg | gccgaggact | gccccgaagt | ccggcacctc |
| 2701 | gtgcacgcgg | atttcggctc | caacaatgtc | ctgacggaca | atggccgcat | aacagcggtc |
| 2761 | attgactgga | gcgaggcgat | gttcggggat | tcccaatacg | aggtcgccaa | catcttcttc |
| 2821 | tggaggccgt | ggttggcttg | tatggagcag | cagacgcgct | acttcgagcg | gaggcatccg |
| 2881 | gagcttgcag | gatcgccgcg | gctccgggcg | tatatgctcc | gcattggtct | tgaccaactc |
| 2941 | tatcagagct | tggttgacgg | caatttcgat | gatgcagctt | gggcgcaggg | tcgatgcgac |
| 3001 | gcaatcgtcc | gatccggagc | cgggactgtc | gggcgtacac | aaatcgcccg | cagaagcgcg |
| 3061 | gccgtctgga | ccgatggctg | tgtagaagta | ctcgccgata | gtggaaaccg | acgccccagc |
| 3121 | actcgtccga | gggcaaagga | atagcacgtg | ctacgagatt | tcgattccac | cgccgccttc |
| 3181 | tatgaaaggt | tgggcttcgg | aatcgttttc | cgggacgccg | gctggatgat | cctccagcgc |
| 3241 | ggggatctca | tgctggagtt | cttcgcccac | cccaacttgt | ttattgcagc | ttataatggt |
| 3301 | tacaaataaa | gcaatagcat | cacaaatttc | acaaataaag | catttttttc | actgcattct |
| 3361 | agttgtggtt | tgtccaaact | catcaatgta | tcttatcatg | tctgtatacc | gtcgacctct |
| 3421 | agctagagct | tggcgtaatc | atggtcatag | ctgtttcctg | tgtgaaattg | ttatccgctc |
| 3481 | acaattccac | acaacatacg | agccggaagc | ataaagtgta | aagcctgggg | tgcctaatga |
| 3541 | gtgagctaac | tcacattaat | tgcgttgcgc | tcactgcccg | ctttccagtc | gggaaacctg |
| 3601 | tcgtgccagc | tgcattaatg | aatcggccaa | cgcgcgggga | gaggcggttt | gcgtattggg |
| 3661 | cgctcttccg | cttcctcgct | cactgactcg | ctgcgctcgg | tcgttcggct | gcggcgagcg |
| 3721 | gtatcagctc | actcaaaggc | ggtaatacgg | ttatccacag | aatcagggga | taacgcagga |
| 3781 | aagaacatgt | gagcaaaagg | ccagcaaaag | gccaggaacc | gtaaaaaggc | cgcgttgctg |
| 3841 | gcgtttttcc | ataggctccg | cccccctgac | gagcatcaca | aaaatcgacg | ctcaagtcag |
| 3901 | aggtggcgaa | acccgacagg | actataaaga | taccaggcgt | ttccccctgg | aagctccctc |
| 3961 | gtgcgctctc | ctgttccgac | cctgccgctt | accggatacc | tgtccgcctt | tctcccttcg |
| 4021 | ggaagcgtgg | cgctttctca | atgctcacgc | tgtaggtatc | tcagttcggt | gtaggtcgtt |
| 4081 | cgctccaagc | tgggctgtgt | gcacgaaccc | cccgttcagc | ccgaccgctg | cgccttatcc |
| 4141 | ggtaactatc | gtcttgagtc | caacccggta | agacacgact | tatcgccact | ggcagcagcc |
| 4201 | actggtaaca | ggattagcag | agcgaggtat | gtaggcggtg | ctacagagtt | cttgaagtgg |
| 4261 | tggcctaact | acggctacac | tagaaggaca | gtatttggta | tctgcgctct | gctgaagcca |
| 4321 | gttaccttcg | gaaaaagagt | tggtagctct | tgatccggca | aacaaaccac | cgctggtagc |
| 4381 | ggtggttttt | ttgtttgcaa | gcagcagatt | acgcgcagaa | aaaaaggatc | tcaagaagat |
| 4441 | cctttgatct | tttctacggg | gtctgacgct | cagtggaacg | aaaactcacg | ttaagggatt |
| 4501 | ttggtcatga | gattatcaaa | aaggatcttc | acctagatcc | ttttaaatta | aaaatgaagt |
| 4561 | tttaaatcaa | tctaaagtat | atatgagtaa | acttggtctg | acagttacca | atgcttaatc |
| 4621 | agtgaggcac | ctatctcagc | gatctgtcta | tttcgttcat | ccatagttgc | ctgactcccc |
| 4681 | gtcgtgtaga | taactacgat | acgggagggc | ttaccatctg | gccccagtgc | tgcaatgata |
| 4741 | ccgcgagacc | cacgctcacc | ggctccagat | ttatcagcaa | taaaccagcc | agccggaagg |
| 4801 | gccgagcgca | gaagtggtcc | tgcaacttta | tccgcctcca | tccagtctat | taattgttgc |
| 4861 | cgggaagcta | gagtaagtag | ttcgccagtt | aatagtttgc | gcaacgttgt | tgccattgct |
| 4921 | acaggcatcg | tggtgtcacg | ctcgtcgttt | ggtatggctt | cattcagctc | cggttcccaa |
| 4981 | cgatcaaggc | gagttacatg | atcccccatg | ttgtgcaaaa | aagcggttag | ctccttcggt |
| 5041 | cctccgatcg | ttgtcagaag | taagttggcc | gcagtgttat | cactcatggt | tatggcagca |
| 5101 | ctgcataatt | ctcttactgt | catgccatcc | gtaagatgct | tttctgtgac | tggtgagtac |
| 5161 | tcaaccaagt | cattctgaga | atagtgtatg | cggcgaccga | gttgctcttg | cccggcgtca |
| 5221 | atacgggata | ataccgcgcc | acatagcaga | actttaaaag | tgctcatcat | tggaaaacgt |
| 5281 | tcttcggggc | gaaaactctc | aaggatotta | ccgctgttga | gatccagttc | gatgtaaccc |
| 5341 | actcgtgcac | ccaactgatc | ttcagcatct | tttactttca | ccagcgtttc | tgggtgagca |
| 5401 | aaaacaggaa | ggcaaaatgc | cgcaaaaaag | ggaataaggg | cgacacggaa | atgttgaata |
| 5461 | ctcatactct | tcctttttca | atattattga | agcatttatc | agggttattg | tctcatgagc |
| 5521 | ggatacatat | ttgaatgtat | ttagaaaaat | aaacaaatag | gggttccgcg | cacatttccc |
| 5581 | cgaaaagtgc | cacctgacgt | c | | | |

The nucleotide sequence for vector pcDNA3.1/Hygro(+) WT E1 (SEQ ID NO: 4) is displayed in Table 5, below.

TABLE 5
Vector pcDNA3.1/Hygro(+) WT E1

| | | | | | | |
|---|---|---|---|---|---|---|
| 1 | gacggatcgg | gagatctccc | gatcccctat | ggtcgactct | cagtacaatc | tgctctgatg |
| 61 | ccgcatagtt | aagccagtat | ctgctccctg | cttgtgtgtt | ggaggtcgct | gagtagtgcg |
| 121 | cgagcaaaat | ttaagctaca | acaaggcaag | gcttgaccga | caattgcatg | aagaatctgc |
| 181 | ttagggttag | gcgttttgcg | ctgcttcgcg | atgtacgggc | cagatatacg | cgttggcaat |
| 241 | cagcttgcta | ctgaaagaca | tttttaggca | ccacgcccag | cgctcctgct | acgactggat |
| 301 | catctacaac | accaccccgc | agcatgaagc | tctgcagtgg | tgctacctcc | accccagaga |
| 361 | cgggcttatg | cccatgtatc | tgaacatcca | gagtcacctt | taccacgtcc | tggaaaaaat |
| 421 | acacaggacc | ctcaacgacc | gagaccgctg | gtcccgggcc | taccgcgcgc | gcaaaacccc |
| 481 | taaataaaga | cagcaagaca | cttgcttgat | ccaaatccaa | acagagtctg | gttttttatt |
| 541 | tatgttttaa | accgcattgg | gaggggagga | agccttcagg | gcagaaacct | gctggcgcag |
| 601 | atccaacagc | tgctgagaaa | cgacattaag | ttcccgggtc | aaagaatcca | attgtgccaa |
| 661 | aagagccgtc | aacttgtcat | cgcgggcgga | tgaacgggaa | gctgcactgc | ttgcaagcgg |
| 721 | gctcaggaaa | gcaaagtcag | tcacaatccc | gcgggcggtg | gctgcagcgg | ctgaagcggc |
| 781 | ggcggaggct | gcagtctcca | acggcgttcc | agacacggtc | tcgtaggtca | aggtagtaga |
| 841 | gtttgcgggc | aggacggggc | gaccatcaat | gctggagccc | atcacattct | gacgcacccc |
| 901 | ggcccatggg | ggcatgcgcg | ttgtcaaata | tgagctcaca | atgcttccat | caaacgagtt |
| 961 | ggtgctcatg | gcggcggcgg | ctgctgcaaa | acagatacaa | aactacataa | gacccccacc |
| 1021 | ttatatattc | tttcccaccc | ttaagccacg | cccacacatt | tcagtacctc | aatctgtatc |
| 1081 | ttcatcgcta | gagccaaact | cagcgcgggt | gcaggccagc | accaagtgat | cgggcctcag |
| 1141 | ctcctcggtc | acatccagca | tcacaggctg | gttcctaata | tgtttaccgc | cacactcgca |
| 1201 | gggtctgcac | ctggtgcggg | tctcatcgta | cctcagcacc | ttccagatct | tcatggtcat |
| 1261 | gtcaaacacc | ccgttcaggt | tcaccttgga | catgctctcg | ggctcaagca | atatcttagt |
| 1321 | gtgactcaaa | ttgcattggt | aaggtaggaa | cacccccctc | ctgttaccca | aatgcaagga |
| 1381 | acagcgggtc | agtatgttat | gctcaaacac | tggccaggcc | ttgcgagagt | ggctggctac |
| 1441 | gtgaatggtc | ttcagcaggt | gacagttgcc | gtccgagcag | gtcagcatct | gagaggccct |
| 1501 | gtcctcgcag | ttgccacata | ccatgttatg | cttaatcaca | gccacgcttt | tcactagcat |
| 1561 | gaagcaacca | cagtcggagg | ccacattgtg | gcgcaccctg | gagttaccct | cagacaggat |
| 1621 | acccaaggta | cacctttcaa | agaggcattt | cttaattgaa | gccctgcttt | tggggcgaca |
| 1681 | caccaccccc | ttccagcagc | agtaaaaggc | acagccccga | acccttacat | cggtccaggc |
| 1741 | ttccacacag | gtattgttaa | acccatagaa | gcttacaccg | tgtaggataa | ggttggtatt |
| 1801 | ggccaggaaa | accgtaccgc | taaaattggg | gccagtaaac | cttacattca | taataaccac |
| 1861 | cccgtccatg | ccaagcaccc | ccggccacat | atttatcatg | ctacatctaa | aggccaccct |
| 1921 | atcctccgta | tctatctcca | cctcggcccc | gttcccagaa | atgtagcaac | aattcctgat |
| 1981 | atttacaagt | ttgctgatct | tgtacttgca | atctggccta | agtgccacct | ttgcatatac |
| 2041 | cctaatagcc | tcctcaaaat | catcccctgg | ctgcagccag | taagtggtca | gctgctctat |
| 2101 | ggaatacttc | tgcgccagca | gatcaagctc | attagcgcaa | ttatccttga | tctgttgaaa |
| 2161 | agtaatacac | tcaggacggt | gtctggtcat | taagctaaaa | gctagattcc | tagcctcctc |
| 2221 | tgtagcctca | caagcccccc | gctccctctt | tacccccttt | agcccctgcc | catcctctgt |
| 2281 | aattgtcaaa | atgcgtctca | gttctggata | cagttcagcc | acctgtacaa | cattcattcc |
| 2341 | cgagggtcca | ggccggctct | cgggttccat | gggctctgct | cctgccgccg | ccgcctggct |
| 2401 | tcctcctgct | gctgctgctg | ctcctccgtc | ggtattatcg | ccgggcggac | ggaagacaac |
| 2461 | agtagcaggc | gattcttgtg | tctcacaacc | gctctccaca | gatgcatggc | cagaaaatcc |
| 2521 | agcaggtacc | ccccgctcag | atgggtttct | tcgctccatt | tatcctttat | aaaactcaaa |
| 2581 | aaagcaacag | cagccgcagc | gcgccccggt | gtggaaaaat | ccaaagtctt | gatgaccttc |
| 2641 | tcttggaaaa | gcgcctggtg | acccagattc | aaagaatcaa | acagctcacc | acaggatttc |
| 2701 | aaaagctctt | caaattccca | cttgtaatcc | tccttaattc | tgcagactaa | ctttgcctgg |
| 2761 | gatgagcccc | acagaaacct | ccaaaaccaa | gaggtactgt | tagagctctg | ttccagcaag |
| 2821 | ttacgcacag | cagaaaaatc | ttccaaacac | tcccaagcct | ccatgaggtc | agatgtaacc |
| 2881 | aagattagcc | cacggcgcat | tatataccct | ttaagccccg | ccccatttaa | cacgccatgc |
| 2941 | aagttaaaca | ttatctcacc | ctttattaaa | cttacatcaa | ctcattcagc | aaacaaaggc |
| 3001 | gttaaccaca | cacgcaatca | caggtttaca | ccttatggcc | tggggcgttt | acagctcaag |
| 3061 | tccaaaggtt | gcccaggctc | gttaagcaag | tcctcgatac | attccacagc | ctggcgacgc |
| 3121 | ccaccaactc | tcacggcaac | tggtttaatg | gggcacagcg | ggaccaccgg | gtgtatctca |
| 3181 | ggaggtgtgt | tagaaggacc | ggagtcacag | ctatccgtac | tactattgca | ttctctagac |
| 3241 | acaggtgatg | tcgggcgtct | caggatagca | ggcgccattt | taggacggcg | ggtaggtctt |
| 3301 | gcaggctccg | gttctggctc | gggctcaggc | tcaggttcag | acacaggacc | ttttaaaaaa |
| 3361 | atcacaatac | aaaattcttt | aaaccacaaa | actgtaaaaa | ttaaaaaaaa | aattaccaca |
| 3421 | ccaaacccac | cactctatca | cccactgccc | ataattttca | cttactgtag | acaaacatgc |
| 3481 | cacaggtcct | catatagcaa | agcgaacaca | taatatctgg | gtcccccgta | ttcctccggt |
| 3541 | gataatgaca | agacctgcaa | ccgtgcccgg | ggtgctccac | ataatctaac | acaaactcct |
| 3601 | caccctcttc | atcctcgtcg | tcactgggtg | gaaagccagc | ctcgtggcag | gtaagatcga |
| 3661 | tcacctccgg | tacaaggttt | ggcatagaaa | ccggacccaa | ggctctctgc | tccggctgct |
| 3721 | cgggctgccg | ggaaaggtga | ggcggctccg | gagaaccggg | cgccggcgga | aaagtgagta |
| 3781 | agtcaatccc | ttcctgcacc | gccaacatta | cagagtcggg | aaaaatctgc | gaaaccgcct |
| 3841 | cctcgttggg | atcttcgggg | gccgtcacgt | ctaaatcata | cagttcgtga | agggtaggtg |
| 3901 | gttcaaaatg | gctaggaggt | ggaagattat | cagccagtac | ctcttcgatc | agctggtcca |
| 3961 | aaagactggc | ggccatttct | tcggtaataa | cacctccgtg | gcagataata | tgtctcattt |
| 4021 | tcagtcccgg | tgtcggagcg | gctcggagga | gaaaactcta | ctcgctggca | ctcaagagtg |
| 4081 | gcctcttgag | gaactcaccg | ggtataaata | cactacacgt | cagctgacta | taataataaa |
| 4141 | acgccaactt | tgacccggaa | cgcggaaaac | acctgagaaa | aacacctggg | cgagtctcca |
| 4201 | cgtaaacggt | caaagtcccc | gcggccctag | acaaatatta | cgcgctatga | gtaacacaaa |
| 4261 | attattcaga | tttcacttcc | tcttattcag | ttttcccgcg | aaaatggcca | aatcttactc |
| 4321 | ggttacgccc | aaatttacta | caacatccgc | ctaaaaccgc | gcgaaaattg | tcacttcctg |
| 4381 | tgtacaccgg | cgcacaccaa | aaacgtcact | tttgccacat | ccgtcgctta | catgtgttcc |
| 4441 | gccacacttg | caacatcaca | cttccgccac | actactacgt | cacccgcccc | gttcccacgc |
| 4501 | cccgcgccac | gtcacaaact | ccaccccctc | attatcatat | tggcttcaat | ccaaaataag |
| 4561 | gtatattatt | gatgatggaa | gacaatagca | ggcatgctgg | ggatgcggtg | ggctctatgg |
| 4621 | cttctgaggc | ggaaagaacc | agctggggct | ctagggggta | tccccacgcg | ccctgtagcg |
| 4681 | gcgcattaag | cgcggcgggt | gtggtggtta | cgcgcagcgt | gaccgctaca | cttgccagcg |
| 4741 | ccctagcgcc | cgctcctttc | gctttcttcc | cttcctttct | cgccacgttc | gccggctttc |
| 4801 | cccgtcaagc | tctaaatcgg | ggcatccctt | tagggttccg | atttagtgct | ttacggcacc |
| 4861 | tcgaccccaa | aaaacttgat | tagggtgatg | gttcacgtag | tgggccatcg | ccctgataga |
| 4921 | cggtttttcg | ccctttgacg | ttggagtcca | cgttctttaa | tagtggactc | ttgttccaaa |
| 4981 | ctggaacaac | actcaaccct | atctcggtct | attcttttga | tttataaggg | attttgggga |
| 5041 | tttcggccta | ttggttaaaa | aatgagctga | tttaacaaaa | atttaacgcg | aattaattct |
| 5101 | gtggaatgtg | tgtcagttag | ggtgtggaaa | gtccccaggc | tccccaggca | ggcagaagta |
| 5161 | tgcaaagcat | gcatctcaat | tagtcagcaa | ccaggtgtgg | aaagtcccca | ggctccccag |
| 5221 | caggcagaag | tatgcaaagc | atgcatctca | attagtcagc | aaccatagtc | ccgcccctaa |
| 5281 | ctccgcccat | cccgccccta | actccgccca | gttccgccca | ttctccgccc | catggctgac |
| 5341 | taattttttt | tatttatgca | gaggccgagg | ccgcctctgc | ctctgagcta | ttccagaagt |
| 5401 | agtgaggagg | cttttttgga | ggcctaggct | tttgcaaaaa | gctcccggga | gcttgtatat |
| 5461 | ccattttcgg | atctgatcag | cacgtgatga | aaaagcctga | actcaccgcg | acgtctgtcg |
| 5521 | agaagtttct | gatcgaaaag | ttcgacagcg | tctccgacct | gatgcagctc | tcggagggcg |
| 5581 | aagaatctcg | tgctttcagc | ttcgatgtag | gagggcgtgg | atatgtcctg | cgggtaaata |
| 5641 | gctgcgccga | tggtttctac | aaagatcgtt | atgtttatcg | gcactttgca | tcggccgcgc |
| 5701 | tcccgattcc | ggaagtgctt | gacattgggg | aattcagcga | gagcctgacc | tattgcatct |
| 5761 | cccgccgtgc | acagggtgtc | acgttgcaag | acctgcctga | aaccgaactg | cccgctgttc |
| 5821 | tgcagccggt | cgcggaggcc | atggatgcga | tcgctgcggc | cgatcttagc | cagacgagcg |
| 5881 | ggttcggccc | attcggaccg | caaggaatcg | gtcaatacac | tacatggcgt | gatttcatat |
| 5941 | gcgcgattgc | tgatccccat | gtgtatcact | ggcaaactgt | gatggacgac | accgtcagtg |
| 6001 | cgtccgtcgc | gcaggctctc | gatgagctga | tgctttgggc | cgaggactgc | cccgaagtcc |
| 6061 | ggcacctcgt | gcacgcggat | ttcggctcca | acaatgtcct | gacggacaat | ggccgcataa |
| 6121 | cagcggtcat | tgactggagc | gaggcgatgt | tcggggattc | ccaatacgag | gtcgccaaca |
| 6181 | tcttcttctg | gaggccgtgg | ttggcttgta | tggagcagca | gacgcgctac | ttcgagcgga |
| 6241 | ggcatccgga | gcttgcagga | tcgccgcggc | tccgggcgta | tatgctccgc | attggtcttg |
| 6301 | accaactcta | tcagagcttg | gttgacggca | atttcgatga | tgcagcttgg | gcgcagggtc |
| 6361 | gatgcgacgc | aatcgtccga | tccggagccg | ggactgtcgg | gcgtacacaa | atcgcccgca |
| 6421 | gaagcgcggc | cgtctggacc | gatggctgtg | tagaagtact | cgccgatagt | ggaaaccgac |
| 6481 | gccccagcac | tcgtccgagg | gcaaaggaat | agcacgtgct | acgagatttc | gattccaccg |
| 6541 | ccgccttcta | tgaaaggttg | ggcttcggaa | tcgttttccg | ggacgccggc | tggatgatcc |
| 6601 | tccagcgcgg | ggatctcatg | ctggagttct | tcgcccaccc | caacttgttt | attgcagctt |
| 6661 | ataatggtta | caaataaagc | aatagcatca | caaatttcac | aaataaagca | tttttttcac |
| 6721 | tgcattctag | ttgtggtttg | tccaaactca | tcaatgtatc | ttatcatgtc | tgtataccgt |
| 6781 | cgacctctag | ctagagcttg | gcgtaatcat | ggtcatagct | gtttcctgtg | tgaaattgtt |
| 6841 | atccgctcac | aattccacac | aacatacgag | ccggaagcat | aaagtgtaaa | gcctggggtg |
| 6901 | cctaatgagt | gagctaactc | acattaattg | cgttgcgctc | actgcccgct | ttccagtcgg |
| 6961 | gaaacctgtc | gtgccagctg | cattaatgaa | tcggccaacg | cgcggggaga | ggcggtttgc |
| 7021 | gtattgggcg | ctcttccgct | tcctcgctca | ctgactcgct | gcgctcggtc | gttcggctgc |
| 7081 | ggcgagcggt | atcagctcac | tcaaaggcgg | taatacggtt | atccacagaa | tcaggggata |
| 7141 | acgcaggaaa | gaacatgtga | gcaaaaggcc | agcaaaaggc | caggaaccgt | aaaaaggccg |
| 7201 | cgttgctggc | gtttttccat | aggctccgcc | cccctgacga | gcatcacaaa | aatcgacgct |
| 7261 | caagtcagag | gtggcgaaac | ccgacaggac | tataaagata | ccaggcgttt | ccccctggaa |
| 7321 | gctccctcgt | gcgctctcct | gttccgaccc | tgccgcttac | cggatacctg | tccgcctttc |
| 7381 | tcccttcggg | aagcgtggcg | ctttctcaat | gctcacgctg | taggtatctc | agttcggtgt |
| 7441 | aggtcgttcg | ctccaagctg | ggctgtgtgc | acgaaccccc | cgttcagccc | gaccgctgcg |
| 7501 | ccttatccgg | taactatcgt | cttgagtcca | acccggtaag | acacgactta | tcgccactgg |
| 7561 | cagcagccac | tggtaacagg | attagcagag | cgaggtatgt | aggcggtgct | acagagttct |
| 7621 | tgaagtggtg | gcctaactac | ggctacacta | gaaggacagt | atttggtatc | tgcgctctgc |
| 7681 | tgaagccagt | taccttcgga | aaaagagttg | gtagctcttg | atccggcaaa | caaaccaccg |
| 7741 | ctggtagcgg | tggttttttt | gtttgcaagc | agcagattac | gcgcagaaaa | aaaggatctc |
| 7801 | aagaagatcc | tttgatcttt | tctacggggt | ctgacgctca | gtggaacgaa | aactcacgtt |
| 7861 | aagggatttt | ggtcatgaga | ttatcaaaaa | ggatottcac | ctagatcctt | ttaaattaaa |
| 7921 | aatgaagttt | taaatcaatc | taaagtatat | atgagtaaac | ttggtctgac | agttaccaat |
| 7981 | gcttaatcag | tgaggcacct | atctcagcga | tctgtctatt | tcgttcatcc | atagttgcct |
| 8041 | gactccccgt | cgtgtagata | actacgatac | gggagggctt | accatctggc | cccagtgctg |
| 8101 | caatgatacc | gcgagaccca | cgctcaccgg | ctccagattt | atcagcaata | aaccagccag |
| 8161 | ccggaagggc | cgagcgcaga | agtggtcctg | caactttatc | cgcctccatc | cagtctatta |
| 8221 | attgttgccg | ggaagctaga | gtaagtagtt | cgccagttaa | tagtttgcgc | aacgttgttg |
| 8281 | ccattgctac | aggcatcgtg | gtgtcacgct | cgtcgtttgg | tatggcttca | ttcagctccg |
| 8341 | gttcccaacg | atcaaggcga | gttacatgat | cccccatgtt | gtgcaaaaaa | gcggttagct |
| 8401 | ccttcggtcc | tccgatcgtt | gtcagaagta | agttggccgc | agtgttatca | ctcatggtta |
| 8461 | tggcagcact | gcataattct | cttactgtca | tgccatccgt | aagatgcttt | tctgtgactg |
| 8521 | gtgagtactc | aaccaagtca | ttctgagaat | agtgtatgcg | gcgaccgagt | tgctcttgcc |
| 8581 | cggcgtcaat | acgggataat | accgcgccac | atagcagaac | tttaaaagtg | ctcatcattg |
| 8641 | gaaaacgttc | ttcggggcga | aaactctcaa | ggatcttacc | gctgttgaga | tccagttcga |
| 8701 | tgtaacccac | tcgtgcaccc | aactgatctt | cagcatcttt | tactttcacc | agcgtttctg |
| 8761 | ggtgagcaaa | aacaggaagg | caaaatgccg | caaaaaaggg | aataagggcg | acacggaaat |
| 8821 | gttgaatact | catactcttc | ctttttcaat | attattgaag | catttatcag | ggttattgtc |
| 8881 | tcatgagcgg | atacatattt | gaatgtattt | agaaaaataa | acaaataggg | gttccgcgca |
| 8941 | catttccccg | aaaagtgcca | cctgacgtc | | | |

The nucleotide sequence for vector pcDNA3.1/Hygro(+) HuPGK E1A E1B bGH (SEQ ID NO: 5) is displayed in Table 6, below.

TABLE 6
Vector pcDNA3.1/Hygro(+) HuPGK E1A E1B bGH

| | | | | | | |
|---|---|---|---|---|---|---|
| 1 | gacggatcgg | gagatctccc | gatcccctat | ggtcgactct | cagtacaatc | tgctctgatg |
| 61 | ccgcatagtt | aagccagtat | ctgctccctg | cttgtgtgtt | ggaggtcgct | gagtagtgcg |
| 121 | cgagcaaaat | ttaagctaca | acaaggcaag | gcttgaccga | caattgcatg | aagaatctgc |
| 181 | ttagggttag | gcgttttgcg | ctgcttcgcg | atgtacgggc | cagatatacg | cgttgacggg |
| 241 | gttggggttg | cgccttttcc | aaggcagccc | tgggtttgcg | cagggacgcg | gctgctctgg |
| 301 | gcgtggttcc | gggaaacgca | gcggcgccga | ccctgggtct | cgcacattct | tcacgtccgt |
| 361 | tcgcagcgtc | acccggatct | tcgccgctac | ccttgtgggc | cccccggcga | cgcttcctgc |
| 421 | tccgccccta | agtcgggaag | gttccttgcg | gttcgcggcg | tgccggacgt | gacaaacgga |
| 481 | agccgcacgt | ctcactagta | ccctcgcaga | cggacagcgc | cagggagcaa | tggcagcgcg |
| 541 | ccgaccgcga | tgggctgtgg | ccaatagcgg | ctgctcagca | gggcgcgccg | agagcagcgg |
| 601 | ccgggaaggg | gcggtgcggg | aggcggggtg | tggggcggta | gtgtgggccc | tgttcctgcc |
| 661 | cgcgcggtgt | tccgcattct | gcaagcctcc | ggagcgcacg | tcggcagtcg | gctccctcgt |
| 721 | tgaccgaatc | accgacctct | ctccccagcc | gggtacgtcg | ctagaggatc | gaacccttgc |
| 781 | caccatgaga | catattatct | gccacggagg | tgttattacc | gaagaaatgg | ccgccagtct |
| 841 | tttggaccag | ctgatcgaag | aggtactggc | tgataatctt | ccacctccta | gccattttga |
| 901 | accacctacc | cttcacgaac | tgtatgattt | agacgtgacg | gcccccgaag | atcccaacga |
| 961 | ggaggcggtt | tcgcagattt | ttcccgactc | tgtaatgttg | gcggtgcagg | aagggattga |
| 1021 | cttactcact | tttccgccgg | cgcccggttc | tccggagccg | cctcaccttt | cccggcagcc |
| 1081 | cgagcagccg | gagcagagag | ccttgggtcc | ggtttctatg | ccaaaccttg | taccggaggt |
| 1141 | gatcgatctt | acctgccacg | aggctggctt | tccacccagt | gacgacgagg | atgaagaggg |
| 1201 | tgaggagttt | gtgttagatt | atgtggagca | ccccgggcac | ggttgcaggt | cttgtcatta |
| 1261 | tcaccggagg | aatacggggg | acccagatat | tatgtgttcg | ctttgctata | tgaggacctg |
| 1321 | tggcatgttt | gtctacagta | agtgaaaatt | atgggcagtg | ggtgatagag | tggtgggttt |
| 1381 | ggtgtggtaa | tttttttttt | aatttttaca | gttttgtggt | ttaaagaatt | ttgtattgtg |
| 1441 | atttttttaa | aaggtcctgt | gtctgaacct | gagcctgagc | ccgagccaga | accggagcct |
| 1501 | gcaagaccta | cccgccgtcc | taaaatggcg | cctgctatcc | tgagacgccc | gacatcacct |
| 1561 | gtgtctagag | aatgcaatag | tagtacggat | agctgtgact | ccggtccttc | taacacacct |
| 1621 | cctgagatac | acccggtggt | cccgctgtgc | cccattaaac | cagttgccgt | gagagttggt |
| 1681 | gggcgtcgcc | aggctgtgga | atgtatcgag | gacttgctta | acgagcctgg | gcaacctttg |
| 1741 | gacttgagct | gtaaacgccc | caggccataa | ggtgtaaacc | tgtgattgcg | tgtgtggtta |
| 1801 | acgcctttt | ttgctgaatg | agttgatgta | agtttaataa | agggtgagat | aatgtttaac |
| 1861 | ttgcatggcg | tgttaaatgg | ggcggggctt | aaagggtata | taatgcgccg | tgggctaatc |
| 1921 | ttggttacat | ctgacctcat | ggaggcttgg | gagtgtttgg | aagatttttc | tgctgtgcgt |
| 1981 | aacttgctgg | aacagagctc | taacagtacc | tcttggtttt | ggaggtttct | gtggggctca |
| 2041 | tcccaggcaa | agttagtctg | cagaattaag | gaggattaca | agtgggaatt | tgaagagctt |
| 2101 | ttgaaatcct | gtggtgagct | gtttgattct | ttgaatctgg | gtcaccaggc | gcttttccaa |
| 2161 | gagaaggtca | tcaagacttt | ggatttttcc | acaccggggc | gcgctgcggc | tgctgttgct |
| 2221 | tttttgagtt | ttataaagga | taaatggagc | gaagaaaccc | atctgagcgg | ggggtacctg |
| 2281 | ctggattttc | tggccatgca | tctgtggaga | gcggttgtga | gacacaagaa | tcgcctgcta |
| 2341 | ctgttgtctt | ccgtccgccc | ggcgataata | ccgacggagg | agcagcagca | gcagcaggag |
| 2401 | gaagccaggc | ggcggcggca | ggagcagagc | ccatggaacc | cgagagccgg | cctggaccct |
| 2461 | cgggaatgaa | tgttgtacag | gtggctgaac | tgtatccaga | actgagacgc | attttgacaa |
| 2521 | ttacagagga | tgggcagggg | ctaaaggggg | taaagaggga | gcggggggct | tgtgaggcta |
| 2581 | cagaggaggc | taggaatcta | gcttttagct | taatgaccag | acaccgtcct | gagtgtatta |
| 2641 | cttttcaaca | gatcaaggat | aattgcgcta | atgagcttga | tctgctggcg | cagaagtatt |
| 2701 | ccatagagca | gctgaccact | tactggctgc | agccagggga | tgattttgag | gaggctatta |
| 2761 | gggtatatgc | aaaggtggca | cttaggccag | attgcaagta | caagatcagc | aaacttgtaa |
| 2821 | atatcaggaa | ttgttgctac | atttctggga | acggggccga | ggtggagata | gatacggagg |
| 2881 | atagggtggc | ctttagatgt | agcatgataa | atatgtggcc | gggggtgctt | ggcatggacg |
| 2941 | gggtggttat | tatgaatgta | aggtttactg | gccccaattt | tagcggtacg | gttttcctgg |
| 3001 | ccaataccaa | ccttatccta | cacggtgtaa | gcttctatgg | gtttaacaat | acctgtgtgg |
| 3061 | aagcctggac | cgatgtaagg | gttcggggct | gtgcctttta | ctgctgctgg | aagggggtgg |
| 3121 | tgtgtcgccc | caaaagcagg | gcttcaatta | agaaatgcct | ctttgaaagg | tgtaccttgg |
| 3181 | gtatcctgtc | tgagggtaac | tccagggtgc | gccacaatgt | ggcctccgac | tgtggttgct |
| 3241 | tcatgctagt | gaaaagcgtg | gctgtgatta | agcataacat | ggtatgtggc | aactgcgagg |
| 3301 | acagggcctc | tcagatgctg | acctgctcgg | acggcaactg | tcacctgctg | aagaccattc |
| 3361 | acgtagccag | ccactctcgc | aaggcctggc | cagtgtttga | gcataacata | ctgacccgct |
| 3421 | gttccttgca | tttgggtaac | aggagggggg | tgttcctacc | ttaccaatgc | aatttgagtc |
| 3481 | acactaagat | attgcttgag | cccgagagca | tgtccaaggt | gaacctgaac | ggggtgtttg |
| 3541 | acatgaccat | gaagatctgg | aaggtgctga | ggtacgatga | gacccgcacc | aggtgcagac |
| 3601 | cctgcgagtg | tggcggtaaa | catattagga | accagcctgt | gatgctggat | gtgaccgagg |
| 3661 | agctgaggcc | cgatcacttg | gtgctggcct | gcacccgcgc | tgagtttggc | tctagcgatg |
| 3721 | aagatacaga | ttgaaagctt | ggtaccgagc | tcggatccac | tagtccagtg | tggtggaatt |
| 3781 | ctgcagatat | ccagcacagt | ggcggccgct | cgagtctaga | gggcccgttt | aaacccgctg |
| 3841 | atcagcctcg | actgtgcctt | ctagttgcca | gccatctgtt | gtttgcccct | cccccgtgcc |
| 3901 | ttccttgacc | ctggaaggtg | ccactcccac | tgtcctttcc | taataaaatg | aggaaattgc |
| 3961 | atcgcattgt | ctgagtaggt | gtcattctat | tctggggggt | ggggtggggc | aggacagcaa |
| 4021 | gggggaggat | tgggaagaca | atagcaggca | tgctggggat | gcggtgggct | ctatggcttc |
| 4081 | tgaggcggaa | agaaccagct | ggggctctag | ggggtatccc | cacgcgccct | gtagcggcgc |
| 4141 | attaagcgcg | gcgggtgtgg | tggttacgcg | cagcgtgacc | gctacacttg | ccagcgccct |
| 4201 | agcgcccgct | cctttcgctt | tcttcccttc | ctttctcgcc | acgttcgccg | gctttccccg |
| 4261 | tcaagctcta | aatcggggca | tccctttagg | gttccgattt | agtgctttac | ggcacctcga |
| 4321 | ccccaaaaaa | cttgattagg | gtgatggttc | acgtagtggg | ccatcgccct | gatagacggt |
| 4381 | ttttcgccct | ttgacgttgg | agtccacgtt | ctttaatagt | ggactcttgt | tccaaactgg |
| 4441 | aacaacactc | aaccctatct | cggtctattc | ttttgattta | taagggattt | tggggatttc |
| 4501 | ggcctattgg | ttaaaaaatg | agctgattta | acaaaaattt | aacgcgaatt | aattctgtgg |
| 4561 | aatgtgtgtc | agttagggtg | tggaaagtcc | ccaggctccc | caggcaggca | gaagtatgca |
| 4621 | aagcatgcat | ctcaattagt | cagcaaccag | gtgtggaaag | tccccaggct | ccccagcagg |
| 4681 | cagaagtatg | caaagcatgc | atctcaatta | gtcagcaacc | atagtcccgc | ccctaactcc |
| 4741 | gcccatcccg | cccctaactc | cgcccagttc | cgcccattct | ccgccccatg | gctgactaat |
| 4801 | tttttttatt | tatgcagagg | ccgaggccgc | ctctgcctct | gagctattcc | agaagtagtg |
| 4861 | aggaggcttt | tttggaggcc | taggcttttg | caaaaagctc | ccgggagctt | gtatatccat |
| 4921 | tttcggatct | gatcagcacg | tgatgaaaaa | gcctgaactc | accgcgacgt | ctgtcgagaa |
| 4981 | gtttctgatc | gaaaagttcg | acagcgtctc | cgacctgatg | cagctctcgg | agggcgaaga |
| 5041 | atctcgtgct | ttcagcttcg | atgtaggagg | gcgtggatat | gtcctgcggg | taaatagctg |
| 5101 | cgccgatggt | ttctacaaag | atcgttatgt | ttatcggcac | tttgcatcgg | ccgcgctccc |
| 5161 | gattccggaa | gtgcttgaca | ttggggaatt | cagcgagagc | ctgacctatt | gcatctcccg |
| 5221 | ccgtgcacag | ggtgtcacgt | tgcaagacct | gcctgaaacc | gaactgcccg | ctgttctgca |
| 5281 | gccggtcgcg | gaggccatgg | atgcgatcgc | tgcggccgat | cttagccaga | cgagcgggtt |
| 5341 | cggcccattc | ggaccgcaag | gaatcggtca | atacactaca | tggcgtgatt | tcatatgcgc |
| 5401 | gattgctgat | ccccatgtgt | atcactggca | aactgtgatg | gacgacaccg | tcagtgcgtc |
| 5461 | cgtcgcgcag | gctctcgatg | agctgatgct | ttgggccgag | gactgccccg | aagtccggca |
| 5521 | cctcgtgcac | gcggatttcg | gctccaacaa | tgtcctgacg | gacaatggcc | gcataacagc |
| 5581 | ggtcattgac | tggagcgagg | cgatgttcgg | ggattcccaa | tacgaggtcg | ccaacatctt |
| 5641 | cttctggagg | ccgtggttgg | cttgtatgga | gcagcagacg | cgctacttcg | agcggaggca |
| 5701 | tccggagctt | gcaggatcgc | cgcggctccg | ggcgtatatg | ctccgcattg | gtcttgacca |
| 5761 | actctatcag | agcttggttg | acggcaattt | cgatgatgca | gcttgggcgc | agggtcgatg |
| 5821 | cgacgcaatc | gtccgatccg | gagccgggac | tgtcgggcgt | acacaaatcg | cccgcagaag |
| 5881 | cgcggccgtc | tggaccgatg | gctgtgtaga | agtactcgcc | gatagtggaa | accgacgccc |
| 5941 | cagcactcgt | ccgagggcaa | aggaatagca | cgtgctacga | gatttcgatt | ccaccgccgc |
| 6001 | cttctatgaa | aggttgggct | tcggaatcgt | tttccgggac | gccggctgga | tgatcctcca |
| 6061 | gcgcggggat | ctcatgctgg | agttcttcgc | ccaccccaac | ttgtttattg | cagcttataa |
| 6121 | tggttacaaa | taaagcaata | gcatcacaaa | tttcacaaat | aaagcatttt | tttcactgca |
| 6181 | ttctagttgt | ggtttgtcca | aactcatcaa | tgtatcttat | catgtctgta | taccgtcgac |
| 6241 | ctctagctag | agcttggcgt | aatcatggtc | atagctgttt | cctgtgtgaa | attgttatcc |
| 6301 | gctcacaatt | ccacacaaca | tacgagccgg | aagcataaag | tgtaaagcct | ggggtgccta |
| 6361 | atgagtgagc | taactcacat | taattgcgtt | gcgctcactg | cccgctttcc | agtcgggaaa |
| 6421 | cctgtcgtgc | cagctgcatt | aatgaatcgg | ccaacgcgcg | gggagaggcg | gtttgcgtat |
| 6481 | tgggcgctct | tccgcttcct | cgctcactga | ctcgctgcgc | tcggtcgttc | ggctgcggcg |
| 6541 | agcggtatca | gctcactcaa | aggcggtaat | acggttatcc | acagaatcag | gggataacgc |
| 6601 | aggaaagaac | atgtgagcaa | aaggccagca | aaaggccagg | aaccgtaaaa | aggccgcgtt |
| 6661 | gctggcgttt | ttccataggc | tccgcccccc | tgacgagcat | cacaaaaatc | gacgctcaag |
| 6721 | tcagaggtgg | cgaaacccga | caggactata | aagataccag | gcgtttcccc | ctggaagctc |
| 6781 | cctcgtgcgc | tctcctgttc | cgaccctgcc | gcttaccgga | tacctgtccg | cctttctccc |
| 6841 | ttcgggaagc | gtggcgcttt | ctcaatgctc | acgctgtagg | tatctcagtt | cggtgtaggt |
| 6901 | cgttcgctcc | aagctgggct | gtgtgcacga | accccccgtt | cagcccgacc | gctgcgcctt |
| 6961 | atccggtaac | tatcgtcttg | agtccaaccc | ggtaagacac | gacttatcgc | cactggcagc |
| 7021 | agccactggt | aacaggatta | gcagagcgag | gtatgtaggc | ggtgctacag | agttcttgaa |
| 7081 | gtggtggcct | aactacggct | acactagaag | gacagtattt | ggtatctgcg | ctctgctgaa |
| 7141 | gccagttacc | ttcggaaaaa | gagttggtag | ctcttgatcc | ggcaaacaaa | ccaccgctgg |
| 7201 | tagcggtggt | ttttttgttt | gcaagcagca | gattacgcgc | agaaaaaaag | gatctcaaga |
| 7261 | agatcctttg | atcttttcta | cggggtctga | cgctcagtgg | aacgaaaact | cacgttaagg |
| 7321 | gattttggtc | atgagattat | caaaaaggat | cttcacctag | atccttttaa | attaaaaatg |
| 7381 | aagttttaaa | tcaatctaaa | gtatatatga | gtaaacttgg | tctgacagtt | accaatgctt |
| 7441 | aatcagtgag | gcacctatct | cagcgatctg | tctatttcgt | tcatccatag | ttgcctgact |
| 7501 | ccccgtcgtg | tagataacta | cgatacggga | gggcttacca | tctggcccca | gtgctgcaat |
| 7561 | gataccgcga | gacccacgct | caccggctcc | agatttatca | gcaataaacc | agccagccgg |
| 7621 | aagggccgag | cgcagaagtg | gtcctgcaac | tttatccgcc | tccatccagt | ctattaattg |
| 7681 | ttgccgggaa | gctagagtaa | gtagttcgcc | agttaatagt | ttgcgcaacg | ttgttgccat |
| 7741 | tgctacaggc | atcgtggtgt | cacgctcgtc | gtttggtatg | gcttcattca | gctccggttc |
| 7801 | ccaacgatca | aggcgagtta | catgatcccc | catgttgtgc | aaaaaagcgg | ttagctcctt |
| 7861 | cggtcctccg | atcgttgtca | gaagtaagtt | ggccgcagtg | ttatcactca | tggttatggc |
| 7921 | agcactgcat | aattctctta | ctgtcatgcc | atccgtaaga | tgcttttctg | tgactggtga |
| 7981 | gtactcaacc | aagtcattct | gagaatagtg | tatgcggcga | ccgagttgct | cttgcccggc |
| 8041 | gtcaatacgg | gataataccg | cgccacatag | cagaacttta | aaagtgctca | tcattggaaa |
| 8101 | acgttcttcg | gggcgaaaac | tctcaaggat | cttaccgctg | ttgagatcca | gttcgatgta |
| 8161 | acccactcgt | gcacccaact | gatcttcagc | atcttttact | ttcaccagcg | tttctgggtg |
| 8221 | agcaaaaaca | ggaaggcaaa | atgccgcaaa | aaagggaata | agggcgacac | ggaaatgttg |
| 8281 | aatactcata | ctcttccttt | ttcaatatta | ttgaagcatt | tatcagggtt | attgtctcat |
| 8341 | gagcggatac | atatttgaat | gtatttagaa | aaataaacaa | ataggggttc | cgcgcacatt |
| 8401 | tccccgaaaa | gtgccacctg | acgtc | | | |

### C. Engineering E1-Complementing BHK Cell Lines

The present disclosure provides for cell lines and methods to produce recombinant adeno-associated virus (rAAV). Specifically, a BHK-21 cell line is transformed with the wild-type (wt) adenoviral E1 gene region or a portion thereof, such that E1 protein is stably expressed in novel BHK-E1 cell lines, as depicted in FIG. 7. The BHK-E1 complement cell lines are then transfected with three plasmids (triple transfection) containing a transgene, AAV2 rep/cap genes, and adenoviral helper genes (FIG. 8), enabling the production of rAAV particles. The BHK-E1 cell lines of the present disclosure are not derived from human aborted fetal tissue, which provides an alternative for rAAV production for those who do not want to use products made using human aborted fetal cell lines.

BHK-21 [C-13] (ATCC #CCL-10) was obtained from the American Type Culture Collection (ATCC, Manassas, VA). The parent line of BHK-21(C-13) was derived from baby hamster kidneys of five unsexed, 1-day-old hamsters in March 1961, by I. A. Macpherson and M. G. P. Stoker. BHK-21 has been used to produce vaccines for animal use (see Pay, T. W., Boge, A., Menard, F. J. & Radlett, P. J. Production of rabies vaccine by an industrial scale BHK 21 suspension cell culture process. Dev Biol Stand 60, 171-4 (1985)) and pharmaceuticals (see Dumont, J., Euwart, D., Mei, B., Estes, S. & Kshirsagar, R. Human cell lines for biopharmaceutical manufacturing: history, status, and future perspectives. Crit Rev Biotechnol 36, 1110-1122 (2016)). BHK-21 is not a human cell line and thus products manufactured using BHK-21 present no ethical issues. Its derivation from mammalian kidney tissue of a young organism may also result in characteristics similar to cells derived from human embryos. Development and expanded use of BHK-21 could provide an ethically acceptable alternative to HEK293 and other cell lines for biopharmaceutical production.

### Example 1

### Cell Culture

BHK-21 was cultured in Dulbecco's Modified Eagle Medium (DMEM) (ATCC, Manassas, VA) supplemented with 10% (v/v) fetal bovine serum (FBS) (Cytiva, Marlborough, MA) and 1% Penicillin-Streptomycin Solution (Pen/Strep) (10,000 IU/mL Penicillin, 10,000 μg/mL Streptomycin) (ATCC, Manassas, VA). For studies, 250,000 BHK-21 cells were plated in 2 mL of DMEM medium containing 10% FBS and 1% Pen/Strep in Corning™ Costar™ Flat Bottom 6-Well Cell Culture Plates (Corning, NY). Cells were incubated at 37° C. in 5% CO₂.

### Plasmids

To determine which genes could impact rAAV production in newly developed cell lines, two versions of “E1 constructs” were developed: 1) a construct containing the exact sequence of a region of HAdV-5 (1-4344 bp of HAdV-5 viral genome) as found in HEK293, wild-type E1 coding sequences (CDS), and 2) a construct with a human phosphoglycerate kinase (HuPGK) promoter and a Kozak sequence replacing the ITR/promoter region, and with the E1A and E1B CDS, followed by a bovine growth hormone polyadenylation (bGH-poly(A)) signal.

The wild-type (wt) nucleotide sequence of the Ad5 E1 gene (from 1 to 4344 bp of the HAdV-5 viral genome) (SEQ ID NO: 1; NCBI (National Center for Biotechnology Information) sequence accession #KF268127), which aligns with that found in the commercially-available HEK293 cell line (ATCC #CRL-1573), was used to produce pcDNA3.1/Hygro(+) WT E1 (FIG. 9, SEQ ID NO: 4). Additional sequence information about human adenovirus available in the NCBI database were used to identify inverted terminal repeat (ITR), E1A, E1B and IX gene sequences and other minor fragment features. That information was used to design a construct, pcDNA3.1/Hygro(+) HuPGK E1A E1B bGH (FIG. 10, SEQ ID NO: 5). Both E1 sequences, wt E1 and HuPGK E1A E1B bGH, were synthesized de novo (GenScript Biotech, USA) based on available nucleotide sequence data.

To create the two “E1 Constructs” described above and in Table 7, below, vector pcDNA3.1/Hygro(+) (SEQ ID NO: 3, FIG. 11) (www.genscript.com/expression-vector-selection-guide.html) was used as a backbone. The pcDNA3.1/Hygro(+) vector carries the selectable markers AmpR (ampicillin resistance for bacteria culture selection) and HygroR (hygromycin resistance for mammalian culture selection). Hygromycin resistance is used to select for mammalian cells that acquire fragments of pDNA that most likely also carry the “E1 Construct”, allowing cell culture growth on selection media containing hygromycin. Cells that did not acquire “E1 Constructs” would generally not be able to proliferate under hygromycin selection.

TABLE 7
E1 Gene and Promoter Variation Groups-E1 Constructs

| E1 Sequence | E1 Construct with E1 Sequence |
|---|---|
| wt E1 CDS | pcDNA3.1/Hygro(+) WT E1 |
| (SEQ ID NO: 1) | (FIG. 9, SEQ ID NO: 4) |
| HuPGK E1A E1B bGH | pcDNA3.1/Hygro(+) HuPGK E1A E1B |
| CDS (SEQ ID NO: 2) | bGH (FIG. 10, SEQ ID NO: 5) |

### Example 2

### Transfection of BHK Cells

Plasmid DNA (4 μg) of the two E1 Constructs, pcDNA3.1/Hygro(+) WT E1 (FIG. 9, SEQ ID NO: 4) and pcDNA3.1/Hygro(+) HuPGK E1A E1B bGH (FIG. 10, SEQ ID NO: 5), were separately added to the BHK-21 cells. Approximately 2.5×10s BHK-21 cells were plated in 2 mL of DMEM media containing 10% FBS and 1% Pen/Strep in Corning™ Costar™ Flat Bottom 6-Well Cell Culture Plates (Corning, NY). Plates were incubated for approximately 48 hours at 37° C. in 5% CO₂. The cells were washed with 1 mL of DPBS 1× (DPBS with calcium and magnesium, Thermo Fisher Scientific, Waltham, MA). Approximately 500 μL of DMEM media containing only 1% Pen/Step (with no FBS) was added to each well and the plates were returned to the CO₂ incubator.

The transfection reagent was prepared as follows. Two sterile 1.5 mL Eppendorf tubes (Corning, NY) were labeled as A and B for dividing amongst the six wells. Approximately 246 μL of DMEM media containing 1% Pen/Strep and 4 μL of pAd5 WT E1 or HuPGK E1A E1B bGH plasmid was added to the first tube, while approximately 246 μL of DMEM media containing 1% Pen/Strep and 4 μL of PEIPro stock solution (PElpro Transfection Reagent REA-245,236 Polyplus, Illkirch-Graffenstaden, France) (1 mg/mL) was added to the second tube. The contents of the two tubes were gently mixed by inverting the tube approximately 10 times and vortexing for 10 seconds. The DNA transfection mix was then incubated at room temperature for about 10 to 15 minutes, but no more than 15 minutes.

The 500 μL of DNA transfection complex was then added to BHK-21 cells in 500 μL of DMEM media containing 1% Pen/Strep. Control cells were maintained throughout the protocol in 1 mL of DMEM media plus 1% Pen/Strep (but no FBS) and 4 μL of PEIPro stock solution. Both transfected and control cells were then incubated at 37° C. in 5% CO₂, and after 72 hours, the media was refreshed with DMEM media containing only 1% Pen/Strep without washing. The cells were then incubated at 37° C. in 5% CO₂ for an additional approximately 48 hours or until the cells reached approximately 80 to 90% confluency. The cells in each well were washed with phosphate buffered saline (DPBS) and fresh growth media containing 35 μg/mL of hygromycin (J607-100MG, VWR, Radnor, PA) was added. The transfected cells were maintained in the media containing hygromycin until the control cells were all dead (typically about 72 hours). The hygromycin resistant cells were collected by trypsinization once they reached confluency and were subcultured in a T75 flask. The cells were incubated at 37° C. in 5% CO2 until they reached confluency.

After an additional 48 hours of incubation, cells were washed with DPBS and 200 p1L of 1× Trypsin-EDTA Solution (ATCC, Manassas, VA) was added per well. The cells were incubated for approximately 5 minutes at 37° C. or until they were completely detached. Then, 9.5 mL of DMEM media containing 10% FBS and 1% Pen/Strep was added and the cells were gently resuspended without centrifugation. Cells were then combined according to experimental group (transfected and control) in T75 flasks (Thermo Fisher Scientific, Waltham, MA). The cells were incubated at 37° C. in 5% CO₂ until they reached confluency. The cells were observed daily for any significant morphological changes in the transfected cells compared to the control cells. Flasks were replenished with fresh media every three days until cells reached a confluency of approximately 70-80%.

Cell viability over time was analyzed by comparing BHK cells transfected with E1 WT plasmid with non-transfected BHK control cells. As shown in FIG. 12, transfected cells had similar viability to control cells for up to 72 hours of culture. Similarly, cell viability over time was analyzed by comparing BHK cells transfected with HuPGK E1A E1B bGH plasmid with non-transfected BHK control cells. As shown in FIG. 13, transfected cells had similar viability to control cells for up to 72 hours of culture.

### Example 3

### Detection of E1A Proteins

Transfected cells were split in a 6-well plate after reaching confluency, along with a non-transfected control. At least 250,000 BHK-21 cells transfected with WT E1 or HuPGK E1A E1B bGH were plated in 2 mL of DMEM growth media containing 35 μg/mL of hygromycin (J607-100MG, VWR, Radnor, PA) and incubated at 37° C. in 5% CO₂. After 48 hours of incubation or once the cells reached 80% confluency, whole cell protein isolation was carried out. Media was removed and the cells were washed with 1 mL of ice-cold PBS. The washed cells were overlaid with RIPA lysis extraction buffer (89901, Thermo Fisher Scientific, Waltham, MA) with protease and phosphatase cocktail (1861281, Thermo Fisher Scientific, Waltham, MA). The cells were collected from the wells and added to 1.5 mL centrifuge tubes by gentle scraping. Collected cells were incubated on ice for approximately 30 minutes, vortexing at high speed every 10 minutes. Protein supernatant was collected after centrifugation at high speed (approximately 14,000 rpm) for 5 minutes at 4° C. The collected supernatant was stored at −80° C.

Total protein estimation was performed using a microplate method and a bicinchoninic acid assay (BCA) protocol known in the art. See www.thermofisher.com/order/catalog/product/23225 or BCA protein assay kit (71285-3, Thermo Fisher Scientific, Waltham, MA) and protocol. Briefly, a bovine serum albumin (BSA) protein standard was prepared using Albumin Standard Ampules, 2 mg/mL (Thermo Fisher Scientific, Waltham, MA) or another commercially available albumin source (See, for example, Goldbio A420-1). The BCA working reagent was prepared at a 1:50 ratio (reagent B: reagent A) according to the manufacturer's instructions based on the volume required for the standards, samples and replicates. Next, 25 μL of each standard and unknown were pipetted into a well of a 96-well plate and 200 μL of working reagent was added to each well. Plates were mixed for approximately 30 seconds using a plate shaker, then covered and incubated at 37° C. for 30 minutes. After cooling to room temperature, absorbance was measured at or near 563 nm using a microplate reader. Concentrations of protein were determined using the BSA standard curve.

Sodium dodecyl sulfate-polyacrylamide gel electrophoresis (SDS-PAGE) was performed according to procedures known in the art. Briefly, the protein samples for loading in the gel were prepared at the ratio of 1:1 in a loading buffer of 2×SDS sample buffer (39000, Biorad, Hercules, CA) containing 50 μL β-mercaptoethanol/mL. The mixed samples were heated at 90° C. for 10 minutes and then loaded into pre-made Criterion TGX Stain Free Precast Gel 4-15% (12 wells, 4568084, Biorad, Hercules, CA), along with 10 μl of protein ladder (Precision Plus Protein Kaleidoscope, 1610375, Biorad, Hercules, CA). The running buffer prepared was a 1× Tris/Glycine/SDS from 10× solution (1610732, Biorad, Hercules, CA) and the protein samples were run at 80 V for 10 minutes, then at 100 V until the loading buffer reached the bottom of gel.

At the end of the run the gel was transferred using a Trans Blot Turbo Transfer System Midi Format 0.2 am PVDF (10017840, Biorad, Hercules, CA) with SDS transfer buffer 1X. The protein transferred to the membrane was washed with TBST (1706435, Biorad, Hercules, CA) for 5 minutes and blocked using 5% BSA for 1 hour at room temperature. The blocked membrane was then washed with TBST for 5 minutes, E1A primary antibody (Sc-25, Santa Cruz Biotechnology, Inc., Dallas, TX) was added and with incubation overnight at 4° C. The next day the primary antibody was removed, and the membrane was washed three times for 10 minutes with TBST. After the primary washing, Mouse IgG secondary antibody (HAF018, Bio Techne R&D Systems, Minneapolis, MN) was added with incubation for 1 hour at room temperature. The membrane was washed with TBST three times for 10 minutes each. The washed membrane was developed by staining with a Pierce ECL Western Blotting Substrate for 1.5 minutes. Results of Western blot analysis of E1A protein production in BHK cells transfected with E1 WT plasmid is shown in FIG. 14A-B, with non-transfected cells as a negative control. Results of Western blot analysis of E1A protein production in BHK cells transfected with HuPGK E1A E1B bGH is shown in FIG. 15A-B, with non-transfected cells as a negative control.

### Example 4

### Production of Recombinant AAV2 by Triple Transfection Method

### Cell Culture

E1-Complementing BHK cells, BHK-[wt E1] and BHK-[HuPGK E1A E1B bGH], were prepared as described above. Cells were cultured in T75 flasks (Thermo Fisher Scientific, Waltham, MA) in DMEM medium (ATCC, Manassas, VA) containing 10% FBS (Cytiva, Marlborough, MA) and 1% Pen/Strep (10,000 IU/mL Penicillin, 10,000 μg/mL Streptomycin) (ATCC, Manassas, VA) and incubated at 37° C. in 5% CO₂ until use.

### Plasmids

Plasmids used for triple transfection are commercially available and obtained from Aldevron, Fargo North Dakota (product web page www.aldevron.com/products/pald-aav). The transgene GFP plasmid, pALD-ITR-GFP, is Aldevron catalog number 5062-10, the rep/cap AAV2 plasmid, pALD-AAV2, is Aldevron catalog number 5057-10 and the helper plasmid, pALD-X80, is Aldevron catalog number 5017-10.

### Triple Transfection of E1-Complementing BHK Cells

Approximately 10×10⁶ BHK-21 and BHK-21 E1 transformed cells were seeded in 175-cm² flasks using 30 mL DMEM supplemented with 10% (v/v) FBS and 1% (v/v) Penicillin/Streptomycin. The flasks were incubated at 37° C. in 5% CO₂ until the cells reached 75-85% confluency. For each flask, two sterile 1.5 mL Eppendorf tubes (Corning, NY) were labeled as A and B for preparing the DNA transfection reagent. In tube A, 221.03 μL of DMEM serum free medium was added, followed by 6.08 μL of rep/cap AAV2, 4.1 μL of transgene GFP, and 18.87 μL of pHelper. In tube B, 163.09 μL of DMEM serum free medium was added, followed by 87.15 μL of PEIPro stock solution (PElpro Transfection Reagent REA-245,236 Polyplus, Illkirch-Graffenstaden, France). The contents of tubes A and B were combined and gently mixed by inverting the tube approximately 10 times and vortexing for approximately 10 seconds. The DNA transfection complex was then incubated at room temperature for no more than 15 minutes.

Cells were washed with 10 mL of DPBS (DPBS with calcium and magnesium, Thermo Fisher Scientific, Waltham, MA) and then 29.5 mL DMEM serum free medium was added to the cells in cell plates. Next, 500 μL of the PElpro/DNA mix was added dropwise to the cells and mixed gently by swirling the plates. The transfected cells were incubated for 24 hours at 37° C. in 5% CO₂. After 24 hours of incubation, 27 mL of media was removed from each flask and the flask was replaced with 27 mL of fresh DMEM serum free medium supplemented with 1% Pen/Strep. The flask was placed back into the incubator for an additional 48 hours at 37° C. in 5% CO₂. After 72 hours, 3.3 mL of 10×AAVX-MAX Lysis Buffer (ThermoFisher catalog number A50520) was added to achieve a final buffer concentration of 1X. Cells were then detached from the flask using a cell scraper and collected in a 150 mL round bottom flask. The flask was placed on a rotating platform and incubated for 2 hours at 37° C. with rotation at 150 rpm. The cell lysate was transferred to 50 mL conical tubes and centrifuged at 4000×g for 30 minutes at 4° C. The supernatant containing the rAAV2 was collected and stored at −80° C. for further purification steps.

Recombinant AAV Production from E1-Complementing BHK Cells

Diluted supernatant samples from triple transfected E1-complementing BHK cells were treated with a buffer containing DNase I and exonuclease. Capsid lysis was performed in a buffer containing Proteinase K using a protocol based on the application note “Optimized in-process recombinant adeno-associated virus (rAAV) vector genome titer protocol using the QIAcuity® Digital PCR System” from Qiagen (published at www.qiagen.com/us/resources/resourcedetail?id=e918c957-bc6e-46f2-bb91-bf67dce88ca7&lang=en) with minor modifications. Treated samples were serially diluted and a QIAcuity One Digital PCR instrument was used to perform amplification. The QIAcuity Probe PCR kit and in-house developed primers targeting pGFP CDS were used to evaluate rAAV produced by cell lines, and SV40 poly(A) region primers were used to evaluate the DNA reference material viral titer. A positive control with known AAV titer and DNA spike were used to spike rAAV and DNA reference material into AAV-negative crude lysate to assess assay performance. The sample dilution buffer used to dilute samples was used as the negative control. The AAV titer established by digital PCR (dPCR) is expressed as the number of viral genomes/mL (vg/mL). For BHK cells transfected with WT E1 and then triple transfected, rAAV2 viral genomes/mL (vg/mL) are reported in FIG. 16.

Crude and purified rAAV samples were tested for the presence of fully assembled viral capsids with use of an AAV2 titration ELISA (PRAAV2R and PRAAV2XP) and Dip‘n’Check AAV2 and AAV3 (PR5223) lateral flow assay accordingly to the manufacturer's protocol (PROGEN, Germany). The tests provide results expressed as the number of capsids/mL. For BHK cells transfected with WT E1 and then triple transfected, rAAV2 capsids/mL are reported in FIG. 17. For BHK cells transfected with HuPGK E1A E1B bGH and then triple transfected, rAAV2 capsids/mL are reported in FIG. 18.

Sodium dodecyl sulfate-polyacrylamide gel electrophoresis (SDS-PAGE) was performed according to procedures known in the art. Briefly, the rAAV samples for loading in the gel were prepared at the ratio of 1:5 in a loading buffer of Lane Marker Reducing Sample Buffer (39000, Thermo Fisher Scientific, Waltham, MA). The mixed samples were heated at 95° C. for 5 minutes and then loaded into pre-made Criterion TGX Stain Free Precast Gel 4-15% (12 wells, 4568084, Biorad, Hercules, CA), along with 10 μL of protein ladder (Precision Plus Protein Kaleidoscope, 1610375, Biorad, Hercules, CA). The running buffer prepared was a 1× Tris/Glycine/SDS from 10× solution (1610732, Biorad, Hercules, CA) and the protein samples were run at 80 V for 10 minutes, then at 100 V until the loading buffer reached the bottom of gel.

The gel was transferred using a Trans Blot Turbo Transfer System Midi Format 0.2 μm PVDF (10017840, Biorad, Hercules, CA) with 1×SDS transfer buffer. The protein transferred to the membrane was washed with TBST (1706435, Biorad, Hercules, CA) and blocked using 5% BSA for 1 hour at room temperature. AAV primary antibody (1:100 dilution in 5% BSA in TBST, 03-61058, American Research Products Inc., Waltham, MA) was added and incubated overnight at 4° C. The next day the primary antibody was removed, and the membrane was washed three times for 10 minutes with TBST. After the primary washing, the membrane was added with Mouse IgG secondary antibody (1:1000 dilution in 5% BSA in TBST, HAF018, Bio Techne R&D Systems, Minneapolis, MN) and incubated for 1 hour at room temperature. After 1 hour of incubation the membrane was washed with TBST three times for 10 minutes each. The washed membrane was developed by staining with a Pierce ECL Western Blotting Substrate for 2 minutes. For BHK cells transfected with WT E1 and then triple transfected, rAAV2 capsid protein (VP1/VP2/VP3) production is shown in FIG. 19A-B. For BHK cells transfected with HuPGK E1A E1B bGH and then triple transfected, rAAV2 capsid protein (VP1/VP2/VP3) production is shown in FIG. 20A-B.

### Example 5

### Detection of the E1 Region of Human Adenovirus 5 in Chromosomal DNA of BHK-[Wt E1]

To establish that BHK-[wt E1] cells have a copy(ies) of the E1 region of hAd5 integrated in chromosomal DNA, rather than transiently expressing E1 from a plasmid or other extrachromosomal site, BHK-[wt E1] cells were passaged multiple times without selection for hygromycin resistance. Genomic DNA (gDNA) from BHK-[wt E1] cells from a third passage in hygromycin-free media, and control cells, was extracted with use of Zymo Quick-DNA Miniprep (D3024, Zymo Research) and gDNA quantity and purity was checked with a spectrophotometer and stored as 20 μL aliquots at −20° C. Genomic DNA was loaded on an agarose gel (0.8%) with ethidium bromide (0.5 μg/mL) and resolved (90 V) on the gel. Fast DNA Ladder (N3238S, New England Biolabs) was used for DNA size markers. High molecular-weight genomic DNA of 10,000 MW or more was extracted from the gel and purified with GeneJET purification kit (K0701, Thermo Scientific). Quality and purity were checked with a spectrophotometer. PCR was performed on the extracted DNA using E1A-specific primers with OneTaq Hot Start 2× MM w/Std Buffer (M0484S, NEB). Fragments of the E1 gene region produced by PCR were identified and resolved on E-GeI™ EX Agarose Gels, 2% (G401002, Invitrogen).

FIG. 21 is an agarose gel electrophoresis of the PCR fragments generated from the high molecular-weight samples, i.e., genomic DNA, using E1 primers. It demonstrates the presence of the E1 gene region produced in BHK-[wt E1] cells (Lane 2). Lane 1 is a negative control demonstrating the absence of E1 in BHK-21 cells. Lane 4 is a negative control in which the sample is water. Lane 3 (HEK293, an E1-complementary cell line) and Lane 5 (plasmid DNA containing the E1 gene region) are positive controls. Lane M is a molecular-weight DNA ladder.

### Example 6

### Production of Recombinant AAV2, AAV5, AAV6 and AAV8 Serotypes in BHK-[Wt E1] by Triple Transfection

### Cell Culture

E1-Complementing BHK cells, BHK-[wt E1], were prepared as described above. Cells were cultured in T75 flasks (Thermo Fisher Scientific, Waltham, MA) in DMEM media (ATCC, Manassas, VA) containing 10% FBS (Cytiva, Marlborough, MA) and 1% Pen/Strep (10,000 U/mL Penicillin, 10,000 μg/mL Streptomycin) (ATCC, Manassas, VA) and incubated at 37° C. in 5% CO₂ until use.

### Plasmids

Plasmids used for triple transfection are commercially available and obtained from Aldevron, Fargo North Dakota (product web page www.aldevron.com/products/pald-aav) and GeneScript, Piscataway, New Jersey. The transgene GFP plasmid, pALD-ITR-GFP, is Aldevron catalog number 5062-10, the rep/cap AAV2, pALD-AAV2, is Aldevron catalog number 5057-10, the rep/cap AAV5, pALD-AAV5, is Aldevron catalog number 5058-10, the rep/cap AAV6, pALD-AAV6, is Aldevron catalog number 5059-10, and the rep/cap AAV8, pAGA-AAV8, is GeneScript catalog number U38SYNPG0-3. The helper plasmid, pALD-HELP, is Aldevron catalog number 5082-10 and was used for AAV2, AAV5, AAV6, and AAV8 transfections.

### Triple Transfection of E1-Complementing BHK-[wt E1] Cells

For each triple transfection, approximately 10×10⁶ BHK-[wt E1] cells were seeded in 175-cm² flasks using 30 mL DMEM supplemented with 10% (v/v) FBS and 1% (v/v) Penicillin/Streptomycin. The flasks were incubated at 37° C. in 5% CO₂ until the cells reached 75-85% confluency. For each flask, two sterile 1.5 mL Eppendorf tubes (Corning, NY) were labeled as A and B for preparing the DNA transfection reagent. Tube A contained three plasmids: 1) the transgene GFP plasmid, 2) the helper plasmid, and 3) an AAV rep/cap plasmid of serotype 2, 5, 6, or 8. The amount of each plasmid was calculated as 1 μg of total DNA per one million cells, with a plasmid molar ratio of 1:1:1 diluted in DMEM serum free medium. Tube B contained PEIPro (PElpro Transfection Reagent REA-245,236 Polyplus, Illkirch-Graffenstaden, France) diluted in DMEM serum free media at a concentration three times higher than the plasmid DNA concentration of Tube A. The contents of tubes A and B were combined and gently mixed by inverting the tube approximately 10 times and vortexing for approximately 10 seconds. The DNA-transfection reagent complex was then incubated at room temperature for at least 10 minutes and no more than 15 minutes.

Before adding the DNA-transfection reagent complex, cells were prepared in serum-free media for transfection. Cells were washed with 10 mL of DPBS (Thermo Fisher Scientific, Waltham, MA) and DMEM serum free media was added to the cells for a concentration of approximately 1×10⁶ cells/mL. The DNA-transfection reagent complex was added dropwise to the cells and mixed gently by swirling the plates. The transfected cells were incubated for 24 hours at 37° C. in 5% CO₂. After 24 hours of incubation, approximately 90% of the media was removed from each flask and replaced with fresh DMEM serum-free media. Cells were incubated for an additional 48 hours at 37° C. in 5% CO₂.

In a separate set of experiments, transfection and post-transfection growth was performed as described above with DMEM 5% (v/v) FBS used in the place of DMEM serum-free media. The main difference between the above protocol using DMEM serum-free media and this set of experiments using DMEM 5% (v/v) FBS was that there was no media change after 24 hours post-transfection. Serum conditions can help increase transfection and AAV yield. See Vandenbergh, L., Xiao, R., Luck, M., Lin, J., Korn, M. and Wilson, J. Efficient Serotype-Dependent Release of Functional Vector into the Culture Medium During Adeno-Associated Virus Manufacturing. Hum. Gene Ther. 21(10): 1251-57 (2010). The production of rAAV2, rAAV5, rAAV6 and rAAV8 in BHK-[wt E1] was measured by ELISA (capsids/mL) and dPCR (viral genomes (vg/mL)) according to methods described above in Example 4. The results demonstrate successful production of rAAV of multiple AAV serotypes in BHK-[wt E1] cells using the triple transfection and post-transfection growth in serum-free media (FIG. 22) and DMEM media containing 5% FBS (FIG. 23).

### Example 7

### Scale-Up Production of Recombinant AAV8-Luciferase in BHK-[Wt E1] by the Triple Transfection Method

### Cell Culture

E1-Complementing BHK cells, BHK-[wt E1], were prepared as described above. Cells were cultured in 5-layer Corning Cell Stack flasks (Thermo Fisher Scientific, Waltham, MA) in DMEM media (ATCC, Manassas, VA) containing 10% FBS (Cytiva, Marlborough, MA) and 1% Pen/Strep (10,000 U/mL Penicillin, 10,000 μg/mL Streptomycin) (ATCC, Manassas, VA) and incubated at 37° C. in 5% CO₂ until use.

### Plasmids

Plasmids used for triple transfection were obtained from Aldevron, Fargo North Dakota (product web page www.aldevron.com/products/pald-aav), GeneScript (Piscataway, New Jersey), and Washington University (St. Louis). The transgene Luc plasmid was provided by Washington Univ., the rep/cap AAV8, pAGA-AAV8, is GeneScript catalog number U38SYNPG0-3, and the helper plasmid, pALD-HELP, is Aldevron catalog number 5082-10.

### Triple Transfection of E1-Complementing BHK-[Wt E1] Cells

For each triple transfection, approximately 2.46×10⁸ BHK-21 E1 transformed cells were seeded on 5-layer Corning Cell Stack flasks using 500 mL of DMEM supplemented with 10% (v/v) FBS and 1% (v/v) Penicillin/Streptomycin. The flasks were incubated for 24 hours at 37° C. in 5% CO₂. For each flask, two sterile 50 mL conical tubes were labeled as A and B for preparing the DNA transfection reagent. Tube A contained three plasmids separately coding for: 1) the transgene Luc, 2) the adenovirus helper genes, and 3) the AAV8 rep/cap genes. The amount of each plasmid was calculated as 1 μg of total DNA per one million cells, with a plasmid molar ratio of 1:1:1 diluted in DMEM serum-free media. Tube B contained PEIPro (PElpro Transfection Reagent REA-245,236 Polyplus, Illkirch-Graffenstaden, France) diluted in DMEM serum-free media at a concentration three times higher than the plasmid DNA concentration of Tube A. The contents of tubes A and B were combined and gently mixed by inverting the tube approximately 10 times. The DNA-transfection reagent complex was incubated at room temperature for at least 10 minutes and no more than 15 minutes.

Before adding the DNA-transfection reagent complex, cells were prepared in 5% FBS (v/v) DMEM media for transfection. Cells were washed with 250 mL of DPBS (Thermo Fisher Scientific, Waltham, MA) and reduced serum (5% FBS) media was added to the cells. Using a 1L sterile bottle, the DNA-transfection reagent complex was added to that bottle, and media from the cell stack was poured into the container to fully mix the complex with the media. All that was then poured back into the cell stack. The transfected cells were incubated for 72 hours at 37° C. in 5% CO₂.

### Example 8

### Scale-Up Production of Recombinant AAV2-Luciferase in BHK-[Wt E1] by the Triple Transfection Method

### Cell Culture

E1-Complementing BHK cells, BHK-[wt E1] were prepared as described above. Cells were cultured in 5-layer Corning Cell Stack flasks (Thermo Fisher Scientific, Waltham, MA) in DMEM medium (ATCC, Manassas, VA) containing 10% FBS (Cytiva, Marlborough, MA) and 1% Pen/Strep (10,000 U/mL Penicillin, 10,000 μg/mL Streptomycin) (ATCC, Manassas, VA) and incubated at 37° C. in 5% CO₂ until use.

### Plasmids

Plasmids used for triple transfection were obtained from Aldevron, Fargo North Dakota (product web page www.aldevron.com/products/pald-aav) and Washington Univ. The transgene Luc plasmid was provided by Washington Univ., the rep/cap AAV2, pALD-AAV2, is Aldevron catalog number 5057-10, and the helper plasmid, pALD-HELP, is Aldevron catalog number 5082-10.

### Triple Transfection of E1-Complementing BHK-[Wt E1] Cells

For each triple transfection, approximately 1.0×10⁸ BHK-[wt E1] transformed cells were seeded on 5-layer Corning Cell Stack flasks using 500 mL of DMEM supplemented with 10% (v/v) FBS and 1% (v/v) Penicillin/Streptomycin. The flasks were incubated for 48 hours at 37° C. in 5% CO₂. For each flask, two sterile 50 mL conical tubes were labeled as A and B for preparing the DNA transfection reagent. Tube A contained three plasmids separately coding for: 1) the transgene Luciferase, 2) the adenovirus helper genes, and 3) AAV2 rep/cap genes. The amount of each plasmid was calculated as 1 μg of total DNA per one million cells, with a plasmid molar ratio of 1:1:1 diluted in DMEM serum-free medium. Tube B contained PEIPro (PElpro Transfection Reagent REA-245,236 Polyplus, Illkirch-Graffenstaden, France) diluted in DMEM serum-free medium at a concentration three times higher than the plasmid DNA concentration of Tube A. The contents of tubes A and B were combined and gently mixed by inverting the tube approximately 10 times. The DNA-transfection reagent complex was incubated at room temperature for at least 10 minutes and no more than 15 minutes.

Before adding the DNA-transfection reagent complex, cells were prepared in 5% FBS (v/v) DMEM media for transfection. Cells were washed with 250 mL of DPBS (Thermo Fisher Scientific, Waltham, MA) and then reduced (5% FBS) serum media was added to the cells. Using a 1L sterile bottle, the DNA-transfection reagent complex was added to that bottle, and media from the cell stack was poured into the container to fully mix the complex with the media. All that was then poured back into the cell stack. The transfected cells were incubated for 72 hours at 37° C. in 5% CO₂.

### Example 9

Harvesting, Purification and Analysis of rAAV2-Luciferase and rAAV8-Luciferase Produced in BHK-[wt E1] Cells
Harvesting of rAAV Particles

For harvesting of rAAV particles produced in serum-free conditions, a lysis method was employed. Briefly, approximately 72 hours after transfection, 10×AAVX-MAX Lysis Buffer (ThermoFisher catalog number A50520) was added to the transfected cells to achieve a final buffer concentration of 1X. Cells were detached from the flask using a cell scraper and collected in a 50 mL conical tube. The tube was placed on a rotating platform and incubated for 2 hours at 37° C. in 5% CO₂. The suspension was vortexed and centrifuged at 4000×g for 30 minutes at 4° C. The supernatant containing the rAAV particles was collected in a new 50 mL conical tube, with aliquots prepared for further analysis.

For harvesting of rAAV particles produced in 5% serum conditions, a freeze-thaw method was employed. Briefly, approximately 72 hours after transfection, the transfected cells were detached from flasks by the addition of 0.5 M EDTA for a final EDTA concentration of 25 mM (small scale) or 50 mM (scale-up). Regarding the cell stacks, EDTA was added to 1L sterile bottle, and media from the flask was poured into that bottle to fully mix EDTA in solution. All that was then poured back into the cell stack. The cells were incubated for 25-30 minutes at 37° C., with tapping of the flasks to encourage full detachment of the cells. For small scale, the suspension was collected in 50 mL conical tubes and centrifuged at 300×g for 10 minutes at 4° C. For scale-up production, the suspension was collected in 1L centrifuge bottles and centrifuged at 300×g for 10 minutes at 4° C. using a large volume centrifuge. The supernatant was collected in a new 50 mL tube or 1L bottle, leaving the cell pellet. The pellet was resuspended in 5 mL (small scale) or 30 mL (scale-up) of PBS-MK buffer (1.3 M NaCl, 1 mM MgCl₂, 2.5 mM KCl in PBS, pH 7.4) and the sample was vortexed to aid in pellet resuspension. The cells were lysed using a freeze-thaw method: incubation in liquid nitrogen, followed by incubation in a 37° C. water bath, and repetition for a total of three freeze-thaw cycles. The lysed pellet was centrifuged for 3000×g for 20 minutes at 4° C. and filtered through 0.22 μM Sartorius 50 mL filters. The cell supernatant that was separated from the cell pellet was filtered using 0.22 μM Sartorius 50 mL (small scale) or 1L (scale up) filters. The rAAV was precipitated by adding 10 g of PEG 8000 (polyethylene glycol) and 5.8 g of NaCl per 100 mL of supernatant and stirred at 4° C. until PEG and NaCl were completely dissolved. The solution was stored overnight at 4° C. The solution was centrifuged at 5000×g for 30 mins at 4° C. and the supernatant was discarded. The pellet was resuspended in PBS-MK buffer (500 mL PBS, 101.66 mg MgCl₂ hexahydrate, 93.2 mg KCl) and combined with cell lysate prepared using freeze thaw.

Purification of rAAV Particles

Purification of rAAV particles was performed using AAVX POROS CaptureSelect (Thermo Fisher Scientific) resin, purchased as pre-packed 1 mL columns (Thermo Fisher Scientific, A36652). Columns were used with AKTA Pure 25 M (Cytiva, 29018226) and the purification process was performed at room temperature (approximately 22° C.). The total protein from cell lysate samples was removed as needed by reducing the pH of cell lysate to pH 4 using HCl. After 30 minutes, the pH was adjusted with NaOH to pH 7 and cell lysate was centrifuged at 4000×g for 30 minutes. Cell lysate was filtered using 0.22 μm filters before being loaded on a column. The column was equilibrated with 4 [CV] of 1×PBS (Cytiva, SH30256.02). Cell lysate application was followed by 20 [CV] of 1×PBS (Cytiva, SH30256.02) as the sample application finish step, and additionally with 6 [CV] of 1×PBS (Cytiva, SH30256.02) as a column wash step. The rAAV were eluted with 3 [CV] of low-pH 50 mM Glycine-HCL buffer, pH 2.7 (Polysciences, 24074-1), and collected as three 1 mL fractions. Collection tubes contained Tris-HCl at 1/10 of the fraction volume. Second and third fractions were combined. The collected rAAV samples were buffer exchanged to 1×PBS+0.001% Pluronic F-68 (Gibco, 24040-032) using Amicon Ultracel-2 mL (Merck Millipore, C86533) and filter sterilized using 0.2 μm syringe filters (Thermo Fisher Scientific, 723-2520).

### Determination of AAV Serotype Identity and Capsid Titer

Purified and crude lysate samples of rAAV2-luciferase and rAAV8-luciferase were tested using a Progen AAV8 and AAV2 Xpress ELISA kit (PRAAV8XP, PRAAV2XP) and AAV Titration ELISA (PRAAV8 and PRAAV2R) with no deviations to the user manual's protocol (available at us.progen.com/AAV/AAV-ELISA/AII-AAV-ELISA-Products/), and results were read on a Synergy HTX Multi-Mode Reader (BioTek, 1341000).

### Determination of Vector Genome Titer

The purified and crude lysate samples rAAV2-Luciferase and rAAV8-Luciferase were diluted to 0.1× concentration in 1× Phosphate Buffered Saline (PBS) (VWR, K813-500ML) containing 0.01% Pluronic F-68 (Gibco, 24040-032) and added to a nucleic acid digestion mixture containing 1× DNase Buffer (New England Biolabs, B0303S), 100U of Deoxyribonuclease|(ThermoFisher, 18047019), 1U of Exonuclease|(ThermoFisher, EN0581), and 0.05% Pluronic F-68; unencapsulated nucleic acid was digested at 37° C. for 1 hour. DNase-resistant particles were lysed at 95° C. for 15 minutes in a solution containing 10 mM EDTA (ThermoFisher, 15575020), 0.55M NaCl and 0.55% Sarkosyl (Teknova, 2P0355). The treated samples were serially diluted in 1×PCR buffer (ThermoFisher, 4486219) containing 0.05% Pluronic F-68 and added to a duplexed dPCR reaction using QIAcuity Probe PCR Kit master mix (Qiagen, 250101); primers and probes were from IDT and target CMV promoter and BGH polyA signal sequence regions of the AAV genome using FAM and ROX fluorophores, respectively, for AAV containing luciferase as the transgene. For AAV containing GFP as the transgene, GFP specific primers and probe with HEX fluorophore were used. Reactions were loaded into a QIAcuity Nanoplate 26K 24-well (Qiagen, 250001) and/or QIAcuity Nanoplate 8.5K 24-well (Qiagen, 250011) and run in a QIAcuity One 5-channel dPCR instrument (Qiagen, 911021). QIAcuity run parameters were default for nanoplate priming and imaging: the onboard thermal cycler profile used an initial denaturation at 95° C. for 15 minutes, followed by 40 cycles of denaturation at 95° C. for 15 seconds, and annealing/extension at 60° C. for 30 seconds.

Determination of the Purity of rAAV2-Luciferase and rAAV8-Luciferase Products

The purified samples of rAAV2-luciferase and rAAV8-luciferase were diluted to 5×10¹¹ capsids/mL in 1×PBS containing Pluronic F-68 and added to NuPAGE LDS Sample Buffer (Invitrogen, NP0008) containing NuPAGE Sample Reducing Agent (Invitrogen, NP0004). A portion of this mixture was denatured at 75° C. for 15 minutes and cooled to room temperature. The other portion was kept at room temperature to demonstrate native protein composition. Both the denatured and native mixtures, containing 7.5×10⁹ total capsids each, were separated at 120V for 1 hour on a NuPAGE 4 to 12% Bis-Tris 1.0 mm Mini Protein Gel (Invitrogen, NP0321BOX) using NuPAGE MOPS running buffer (Invitrogen, NP0001) with NuPAGE Antioxidant (Invitrogen, NP0005) in a Mini Gel Tank (Invitrogen, A25977). A Mark12 Unstained Standard Protein Standard (Invitrogen, LC5677) was included for molecular weight sizing. Results were visualized using SilverXpress Silver Staining Kit (Invitrogen LC6100) and imaged with an Azure C300 imager. Densitometry was performed using AzureSpot Pro software.

### Determination of AAV Capsid Identity and Ratio of Capsid Proteins by Western Blotting

The purified samples of rAAV2-luciferase and rAAV8-luciferase were diluted to 2×10¹¹ capsids/mL and added to NuPAGE LDS Sample Buffer (Invitrogen, NP0008) containing NuPAGE Sample Reducing Agent (Invitrogen, NP0004). A portion of this mixture was denatured at 75° C. for 15 minutes and then cooled to room temperature. This mixture, containing 1×10⁹ total capsids, was separated at 120V for 1 hour on a NuPAGE 4 to 12% Bis-Tris 1.0 mm Mini Protein Gel (Invitrogen, NP0323BOX) using NuPAGE MOPS running buffer (Invitrogen, NP0001) with NuPAGE Antioxidant (Invitrogen, NP0005) in a Mini Gel Tank (Invitrogen, A25977). A Precision Plus Protein Kaleidoscope Prestained Protein Standard (BioRad, 1610375) was included for molecular-weight sizing. After SDS-PAGE, the gel was transferred to a 0.45 μM PVDF Membrane (Invitrogen, LC2005) at 20V for 1 hour in a Blot Module (Invitrogen, B1000). The membrane was blocked with 1×TBS (BioRad, 1706436) containing 0.1% Tween-20 (Sigma Aldrich, P9416-100ML), and 5% BSA (GoldBio, A-420-1) at room temperature for 1 hour and stained with an Anti-AAV VP1/VP2/VP3 primary antibody (American Research Products, 03-65158) in the aforementioned buffer overnight at 4° C. After 3 washes in 1×TBST, the membrane was stained with an Anti-Mouse Secondary antibody (R&D Systems, HAF007) in 1×TBST buffer with 5% BSA at room temperature for 1 hour. After 6 washes in 1×TBST, the membrane was developed for 1 minute using the Pierce ECL Western Blotting Substrate Kit (Thermo Scientific, 32106). Results were visualized using an Azure C300 Chemiluminescence Imager. Densitometry was performed using AzureSpot Pro software.

Determination of rAAV2 and rAAV8 Titer

Scaled-up production of recombinant AAV particles was measured by ELISA (capsids/mL) and dPCR (viral genomes (vg/mL)) from crude lysate and purified lysate of BHK-[wt E1] cells, as described in detail above. Results for production of rAAV8-luciferase particles are reported in FIG. 24 and results for rAAV2-luciferase particles are reported in FIG. 25.

### Example 10

### Infectivity in HepG2 Cells of RAAV2-Luciferase and RAAV8-Luciferase Produced in BHK-[wt E1]

### Cells

HepG2 cells were cultured at 25,000 cells/100 μL in 96-well plates and incubated for 48 hours at 37° C. and 5% CO₂. Next, 10-fold serial dilutions of rAAV2 luciferase or rAAV8 luciferase vectors were prepared in BHK-[wt E1] and HepG2 culture media, with dilutions of 2×10¹⁰ vg/mL, 2×10⁹ vg/mL, 2×10⁸ vg/mL, and 2×10⁷ vg/mL. The media was removed from the cells, followed by a wash with 50 μL DPBS and the addition of each dilution or control in duplicate or triplicate. The well plates were incubated for 48 hours at 37° C. in 5% CO₂, after which the cells were lysed and the luciferase activity of the lysate was quantified using a Bright-Glo luciferase assay system (Promega Cat #E2610, Madison WI).

Briefly, cells were equilibrated to room temperature prior to lysis and media was aspirated from the wells. Cells were gently washed with PBS, followed by the addition of 200 μL of Glo lysis buffer. The well plates were rocked slowly to ensure coverage of the cells with the lysis buffer and incubated at room temperature for approximately 5 minutes. Next, 100 μL of the lysate was transferred to 96-well plates for luminescence to be measured.

Infectivity of rAAV particles purified from BHK-[wt E1] cells was demonstrated by measuring luciferase activity from HepG2 cells infected with rAAV8-luciferase (FIG. 26) and rAAV2-luciferase (FIG. 27).

The BHK-[wt E1] cell line was deposited with the American Type Culture Collection (ATCC) on Feb. 14, 2023 as Patent Deposit Number PTA-127522. The BHK-[HuPGK E1A E1B bGH] cell line was deposited with the ATCC on Feb. 14, 2023 as Patent Deposit Number PTA-127523.

As will be understood by those familiar with the art, the present invention may be embodified in other specific forms without departing from the spirit or other essential characteristics thereof. Accordingly, the disclosures and descriptions herein are intended to be illustrative, but not limiting, of the scope of the invention which is set forth in the following claims.

We claim:

1. A recombinant BHK-21 cell line capable of producing a recombinant adeno-associated virus, and designated BHK-[wt E1], deposited on Feb. 14, 2023 at the American Type Culture Collection, Manassas, Virginia under Patent Deposit Number PTA-127522.

2. A recombinant BHK-21 cell line capable of producing a recombinant adeno-associated virus, designated BHK-[HuPGK E1A E1B bGH], deposited on Feb. 14, 2023 at the American Type Culture Collection, Manassas, Virginia under Patent Deposit Number PTA-127523.

3. A recombinant BHK-21 cell line comprising a functional E1 gene region of human adenovirus.

4. The recombinant BHK-21 cell line of claim 3, wherein the functional E1 gene region is the wild-type E1 gene of human adenovirus 5.

5. The recombinant BHK-21 cell line of claim 3, wherein the functional E1 gene comprises positions 1-4344 of a nucleic acid sequence having at least 90% sequence identity with SEQ ID NO: 1.

6. The recombinant BHK-21 cell line of claim 3, capable of producing a recombinant adeno-associated virus (rAAV).

7. The recombinant BHK-21 cell line of claim 6, wherein the rAAV comprises a transgene.

8. The recombinant BHK-21 cell line of claim 3, capable of producing a recombinant adeno-associated virus (rAAV) that comprises a transgene upon transfection with exogenous nucleic acid comprising genes for AAV rep/cap proteins, genes for helper proteins and the transgene.

9. The recombinant BHK-21 cell line of claim 8, wherein the exogenous nucleic acid comprises three vectors wherein a first vector encodes the genes for AAV rep/cap proteins, a second vector encodes genes for helper proteins and a third vector encodes the transgene.

10. The recombinant BHK-21 cell line of claim 9, wherein one or more of the AAV rep/cap proteins are AAV serotype 2, AAV serotype 5, AAV serotype 6, AAV serotype 8, a naturally occurring serotype, an artificial serotype, or a combination of two or more of the foregoing.

11. A method of making a recombinant BHK-21 cell line that is capable of producing a recombinant adeno-associated virus (rAAV) comprising transfecting BHK-21 cells with a vector comprising a functional E1 gene region of human adenovirus.

12. The method of claim 11, wherein the functional E1 gene comprises positions 1-4344 of a nucleic acid sequence having at least 90% sequence identity with SEQ ID NO: 1.

13. The method of claim 12, wherein the vector further comprises a selectable marker.

14. A method of producing a polypeptide comprising: transfecting a BHK-21 cell line comprising a functional E1 gene region of human adenovirus with exogenous nucleic acid comprising genes for AAV rep/cap proteins, genes for helper proteins and a transgene; harvesting rAAV particles comprising the transgene; infecting host cells with the harvested rAAV comprising the transgene; and incubating the host cells to allow production of a polypeptide encoded by the transgene.

15. The method of claim 14, wherein the exogenous nucleic acid comprises three vectors wherein a first vector encodes the genes for AAV rep/cap proteins, a second vector encodes genes for helper proteins and a third vector encodes the transgene.

16. The method of claim 15, wherein one or more of the AAV rep/cap proteins are AAV serotype 2, AAV serotype 5, AAV serotype 6, AAV serotype 8, a naturally-occurring serotype, an artificial serotype, or a combination of two or more of the foregoing.

17. The method of claim 15, wherein the host cell is an animal cell.

18. The method of claim 15, wherein the host cell is HepG2.

19. The method of claim 15, wherein the polypeptide is a pharmaceutical product that provides a therapeutic benefit to an animal.

20. The method of claim 15, wherein the BHK-21 cell line comprising a functional E1 gene region of human adenovirus is BHK-[wt E1], deposited on Feb. 14, 2023 at the American Type Culture Collection, Manassas, Virginia under Patent Deposit Number PTA-127522.


SEQUENCE LISTING (5 sequences)

SEQ ID NO: 1 - DNA, 4344 bp, Human mastadenovirus C
  source 1..4344: mol_type = genomic DNA
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgcca

SEQ ID NO: 2 - DNA, 3839 bp, synthetic construct
  source 1..3839: mol_type = other DNA
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagccgggtacgtcgctagaggatcgaacccttgccaccatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 3 - DNA, 5601 bp, synthetic construct
  source 1..5601: mol_type = other DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 4 - DNA, 8969 bp, synthetic construct
  source 1..8969: mol_type = other DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttggcaatcagcttgctactgaaagacatttttaggcaccacgcccagcgctcctgctacgactggatcatctacaacaccaccccgcagcatgaagctctgcagtggtgctacctccaccccagagacgggcttatgcccatgtatctgaacatccagagtcacctttaccacgtcctggaaaaaatacacaggaccctcaacgaccgagaccgctggtcccgggcctaccgcgcgcgcaaaacccctaaataaagacagcaagacacttgcttgatccaaatccaaacagagtctggttttttatttatgttttaaaccgcattgggaggggaggaagccttcagggcagaaacctgctggcgcagatccaacagctgctgagaaacgacattaagttcccgggtcaaagaatccaattgtgccaaaagagccgtcaacttgtcatcgcgggcggatgaacgggaagctgcactgcttgcaagcgggctcaggaaagcaaagtcagtcacaatcccgcgggcggtggctgcagcggctgaagcggcggcggaggctgcagtctccaacggcgttccagacacggtctcgtaggtcaaggtagtagagtttgcgggcaggacggggcgaccatcaatgctggagcccatcacattctgacgcaccccggcccatgggggcatgcgcgttgtcaaatatgagctcacaatgcttccatcaaacgagttggtgctcatggcggcggcggctgctgcaaaacagatacaaaactacataagacccccaccttatatattctttcccacccttaagccacgcccacacatttcagtacctcaatctgtatcttcatcgctagagccaaactcagcgcgggtgcaggccagcaccaagtgatcgggcctcagctcctcggtcacatccagcatcacaggctggttcctaatatgtttaccgccacactcgcagggtctgcacctggtgcgggtctcatcgtacctcagcaccttccagatcttcatggtcatgtcaaacaccccgttcaggttcaccttggacatgctctcgggctcaagcaatatcttagtgtgactcaaattgcattggtaaggtaggaacacccccctcctgttacccaaatgcaaggaacagcgggtcagtatgttatgctcaaacactggccaggccttgcgagagtggctggctacgtgaatggtcttcagcaggtgacagttgccgtccgagcaggtcagcatctgagaggccctgtcctcgcagttgccacataccatgttatgcttaatcacagccacgcttttcactagcatgaagcaaccacagtcggaggccacattgtggcgcaccctggagttaccctcagacaggatacccaaggtacacctttcaaagaggcatttcttaattgaagccctgcttttggggcgacacaccacccccttccagcagcagtaaaaggcacagccccgaacccttacatcggtccaggcttccacacaggtattgttaaacccatagaagcttacaccgtgtaggataaggttggtattggccaggaaaaccgtaccgctaaaattggggccagtaaaccttacattcataataaccaccccgtccatgccaagcacccccggccacatatttatcatgctacatctaaaggccaccctatcctccgtatctatctccacctcggccccgttcccagaaatgtagcaacaattcctgatatttacaagtttgctgatcttgtacttgcaatctggcctaagtgccacctttgcatataccctaatagcctcctcaaaatcatcccctggctgcagccagtaagtggtcagctgctctatggaatacttctgcgccagcagatcaagctcattagcgcaattatccttgatctgttgaaaagtaatacactcaggacggtgtctggtcattaagctaaaagctagattcctagcctcctctgtagcctcacaagccccccgctccctctttaccccctttagcccctgcccatcctctgtaattgtcaaaatgcgtctcagttctggatacagttcagccacctgtacaacattcattcccgagggtccaggccggctctcgggttccatgggctctgctcctgccgccgccgcctggcttcctcctgctgctgctgctgctcctccgtcggtattatcgccgggcggacggaagacaacagtagcaggcgattcttgtgtctcacaaccgctctccacagatgcatggccagaaaatccagcaggtaccccccgctcagatgggtttcttcgctccatttatcctttataaaactcaaaaaagcaacagcagccgcagcgcgccccggtgtggaaaaatccaaagtcttgatgaccttctcttggaaaagcgcctggtgacccagattcaaagaatcaaacagctcaccacaggatttcaaaagctcttcaaattcccacttgtaatcctccttaattctgcagactaactttgcctgggatgagccccacagaaacctccaaaaccaagaggtactgttagagctctgttccagcaagttacgcacagcagaaaaatcttccaaacactcccaagcctccatgaggtcagatgtaaccaagattagcccacggcgcattatataccctttaagccccgccccatttaacacgccatgcaagttaaacattatctcaccctttattaaacttacatcaactcattcagcaaacaaaggcgttaaccacacacgcaatcacaggtttacaccttatggcctggggcgtttacagctcaagtccaaaggttgcccaggctcgttaagcaagtcctcgatacattccacagcctggcgacgcccaccaactctcacggcaactggtttaatggggcacagcgggaccaccgggtgtatctcaggaggtgtgttagaaggaccggagtcacagctatccgtactactattgcattctctagacacaggtgatgtcgggcgtctcaggatagcaggcgccattttaggacggcgggtaggtcttgcaggctccggttctggctcgggctcaggctcaggttcagacacaggaccttttaaaaaaatcacaatacaaaattctttaaaccacaaaactgtaaaaattaaaaaaaaaattaccacaccaaacccaccactctatcacccactgcccataattttcacttactgtagacaaacatgccacaggtcctcatatagcaaagcgaacacataatatctgggtcccccgtattcctccggtgataatgacaagacctgcaaccgtgcccggggtgctccacataatctaacacaaactcctcaccctcttcatcctcgtcgtcactgggtggaaagccagcctcgtggcaggtaagatcgatcacctccggtacaaggtttggcatagaaaccggacccaaggctctctgctccggctgctcgggctgccgggaaaggtgaggcggctccggagaaccgggcgccggcggaaaagtgagtaagtcaatcccttcctgcaccgccaacattacagagtcgggaaaaatctgcgaaaccgcctcctcgttgggatcttcgggggccgtcacgtctaaatcatacagttcgtgaagggtaggtggttcaaaatggctaggaggtggaagattatcagccagtacctcttcgatcagctggtccaaaagactggcggccatttcttcggtaataacacctccgtggcagataatatgtctcattttcagtcccggtgtcggagcggctcggaggagaaaactctactcgctggcactcaagagtggcctcttgaggaactcaccgggtataaatacactacacgtcagctgactataataataaaacgccaactttgacccggaacgcggaaaacacctgagaaaaacacctgggcgagtctccacgtaaacggtcaaagtccccgcggccctagacaaatattacgcgctatgagtaacacaaaattattcagatttcacttcctcttattcagttttcccgcgaaaatggccaaatcttactcggttacgcccaaatttactacaacatccgcctaaaaccgcgcgaaaattgtcacttcctgtgtacaccggcgcacaccaaaaacgtcacttttgccacatccgtcgcttacatgtgttccgccacacttgcaacatcacacttccgccacactactacgtcacccgccccgttcccacgccccgcgccacgtcacaaactccaccccctcattatcatattggcttcaatccaaaataaggtatattattgatgatggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 5 - DNA, 8425 bp, synthetic construct
  source 1..8425: mol_type = other DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagccgggtacgtcgctagaggatcgaacccttgccaccatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc